SEQUENCE LISTING (2 sequences)

SEQ ID NO: 1 - DNA, 50 bp, synthetic construct
  source 1..50: mol_type = other DNA
  variation 42: note = n = any of C, G, T, A, or U
tcgtcggcagcgtcagatgtgtataagagacagcctacgggnggcwgcag

SEQ ID NO: 2 - DNA, 55 bp, synthetic construct
  source 1..55: mol_type = other DNA
gtctcgtgggctcggagatgtgtataagagacaggactachvgggtatctaatcc

(19) United States
(12) Patent Application Publication
(10) Pub. No.: US 2025/0303006 A1
(43) Pub. Date: Oct. 2, 2025

(54) BIOCOMPATIBLE ANTISEPTIC COMPOSITION CONTAINING 9-HYDROXYCALABAXANTHONE AND/OR RELATED XANTHONES

(71) Applicants: UNIVERSITAT DE LES ILLES BALEARS, Palma de Mallorca (ES); FUNDACIÓ INSTITUT D'INVESTIGACIÓ SANITÀRIA ILLES BALEARS (IDISBA), Palma de Mallorca (ES)

(72) Inventors: Marta MONJO CABRER, Palma de Mallorca (ES); Joana Maria RAMIS MOREY, Palma de Mallorca (ES); Nelson VARGAS ALFREDO, Palma de Mallorca (ES); Marta MUNAR BESTARD, Palma de Mallorca (ES)

(21) Appl. No.: 18/850,372

(22) PCT Filed: Mar. 24, 2023
(86) PCT No.: PCT/EP2023/057726

§ 371 Date: Sep. 24, 2024

(30) Foreign Application Priority Data
Mar. 25, 2022 (EP) 22382276.8

(51) Int. Cl. A61L 2/00; A61K 9/06; A61K 31/353; A61K 47/36

(52) U.S. Cl. CPC A61L 2/0082; A61K 9/06; A61K 31/353; A61K 47/36

(57) ABSTRACT

Biocompatible antiseptic composition containing 9-hydroxycalabaxanthone and/or related xanthones The present invention relates to a cosmetic or pharmaceutical composition containing 9-hydroxycalabaxanthone in the form of gel, aqueous solution, soap, or other presentations, which can be used efficiently in the antiseptic treatment of the buccal cavity, as well as in the disinfection of surfaces such as body surfaces, for example, in the treatment of medical conditions associated with infection (such as acute or chronic wounds, burns, and surgical wounds), or disinfection of the skin before a surgical procedure.

### REFERENCE TO AN ELECTRONIC SEQUENCE LISTING

The contents of the electronic sequence listing (920216_402USPC_SeqListing.xml; Size: 3,524 bytes; and Date of Creation: Sep. 24, 2024) is herein incorporated by reference in its entirety.

### FIELD OF THE ART

The present invention relates to a cosmetic or pharmaceutical composition containing 9-hydroxycalabaxanthone in the form of gel, aqueous solution, soap, or other presentations, which can be used efficiently in the antiseptic treatment of the buccal cavity, as well as in the disinfection of surfaces such as body surfaces.

### BACKGROUND OF THE INVENTION

Iodine, chlorhexidine, alcohol, acetate, hydrogen peroxide, boric acid, silver nitrate, silver sulfadiazine, and sodium hypochlorite are among the most widely used commercialized antiseptic agents in clinical practice. However, despite being excellent antiseptics, many of them present significant biocompatibility issues when they come into contact with living tissues.

In the dental field, there are many products on the market today for the treatment of the buccal cavity, and more specifically for the prevention and treatment of periodontitis, gingivitis, and for antiseptic protection after periodontal and/or peri-implant surgeries, after tooth extractions, or after the placement of dental implants. These formulations mainly consist of a physical mixture between high molecular weight natural substances for forming gels and an active substance with antimicrobial properties. Additionally, regenerative and/or anti-inflammatory properties are sought in many products. The most widely used products are hyaluronic acid (HA) as a gelling substance which also promotes tissue regeneration and chlorhexidine (CHX) as an antimicrobial substance, among others. These systems work suitably under certain conditions; however, they have some drawbacks. On one hand, the main drawbacks result from the very nature of the manufacture thereof. Since they are gels formed solely by means of the physical interaction of polymer chains, i.e., formed by means of physical gelling, they are very unstable in aqueous media. This results in frequent dosing of the treatment and the inability to use same as a prolonged release system. On the other hand, another characteristic of some of these systems is that they depend on CHX or other substances as the antimicrobial active ingredient. Although CHX has excellent antimicrobial properties, harmful effects on human gingival tissues have recently been reported.

Furthermore, it has other side effects which also limit its use including, among them, impairment of the sense of taste, staining of teeth, and even peeling of oral mucosa.

For this reason, it is important to have a better alternative which has regenerative and antimicrobial properties without cytotoxic effects on human cells, and which is being released in a controlled and prolonged manner at the site of action.

### BRIEF DESCRIPTION OF THE FIGURES

FIG. 1. Chemical structure of 9-hydroxycalabaxanthone (HCX).

FIG. 2. Effect of different concentrations of 9-hydroxycalabaxanthone (HCX) and 0.001% of chlorhexidine (CHX) on the activity of LDH released into the medium (A), metabolic activity (B), and morphology (C) of gingival fibroblasts. *p<0.05 treatment vs. C−; ^{#}p<0.05 treatment vs. CHX; ^{&}p<0.05 treatment vs. 0.002% HCX.

FIG. 3. Effect of different concentrations of 9-hydroxycalabaxanthone (HCX) and 0.001% of chlorhexidine (CHX) on the growth (FIG. 3a) and live/dead ratio (FIG. 3b) of P. gingivalis. *p<0.05 treatment vs. C−; ^{#}p<0.05 treatment vs. CHX; ^{&}p<0.05 treatment vs. 0.002%.

FIG. 4. Characterization by means of ¹H NMR in deuterated water (D₂O) at 300 MHz of the chemical modification of HA with adipic acid dihydrazide (ADH). (A) HA before modification and (B) obtaining HA-ADH.

FIG. 5. Characterization by means of ¹H NMR in deuterated water (D₂O) at 300 MHz of the chemical modification of HA with aldehyde groups (CHO). (A) HA before modification and (B) obtaining HA-CHO.

FIG. 6. (A) Characterization of the swelling of modified HA gels and (B) cumulative release of HCX from a HA gel with 0.05% mM HCX.

FIG. 7. Effect of modified HA gels with different concentrations of HCX on the growth (FIG. 7a) and live/dead ratio (FIG. 7b) of P. gingivalis. *p<0.05 treatment vs. HA; ^{#}p<0.05 treatment vs. HA 0.2% CHX; ^{$}p<0.05 treatment vs HA 0.025% HCX.

FIG. 8. MTT viability assay in 3D gingival tissue after the application of HA gels with different concentrations of HCX. *p<0.05 vs C−; ^{$}p<0.05 vs SDS 5%; ⁺p<0.05 vs HA; ^{#}p<0.05 vs HA 0.2% CHX.

FIG. 9. Antimicrobial activity of different concentrations of 9-hydroxycalabaxanthone (HCX) and 0.001% of chlorhexidine (CHX) (A) S. epidermidis growth rate cultured for 10 h with different concentrations of HCX. Results are expressed as % vs negative control that was set to 100%. (B) S. epidermidis growth rate cultured for 24 h with different concentrations of HCX. Results are expressed as % vs negative control that was set to 100%. (C) S. epidermidis Live/Dead Ratio cultured for 24 h with different concentrations of HCX. Data represent the mean±SEM (n=6). Negative control (C−) was bacterial suspension without any treatment. Results were statistically compared by ANOVA and Bonferroni as post hoc: *p<0.05 Treatment vs. C−. ⁺p<0.05 Treatment vs. 0.001% CHX. ^{\hat{ }}p<0.05 Treatment vs. 0.003% HCX. ^{@}p<0.05 Treatment vs 0.002% HCX. ^{%}p<0.05 Treatment vs. 0.001% HCX.

FIG. 10. Oral microbial community in healthy periodontium (Control) and periodontitis (comparison 1). A Composition of oral microbial community at phylum level.

FIG. 11. Relative abundance of phyla. Oral microbial community in treatment A (animals without treatment), treatment B (animals with HA+0.05% HCX gel treatment) and treatment C (animals with HA+0.2% CHX gel treatment) (comparison 2).

FIG. 12. Relative abundance (%) of the identified family in animals with treatment A (animals without treatment), treatment B (animals with HA with 0.05% HCX gel treatment) and treatment C (animals with HA with 0.2% CHX (commercial gel) treatment). (A) Relative abundance (%) of Enterobacteriaceae. (B) Relative abundance (%) of Sphingomonadaceae. p values (<0.05) were obtained using Kruskal Wallis+Conover's test: *p<0.05 Treatment vs. treatment A. #p<0.05 Treatment vs. treatment B.

FIG. 13. Antimicrobial activity of different concentration of HCX (A) S. aureus growth rate cultured at 3 h with different concentrations of chlorhexidine (CHX) and 9-Hydroxycalabaxanthone (HCX). (C) S. aureus Live/Dead Ratio cultured for 3 h with different concentrations of CHX and HCX. Data represent the mean±SEM. The negative control (C−) was bacterial suspension without any treatment. Results were statistically compared by ANOVA and Bonferroni as post hoc: *p<0.05 Treatment vs. C−. ^{#}p<0.05 Treatment vs. 0.01% CHX. ^{&}p<0.05 Treatment vs. 0.005% CHX. ^{$}p<0.05 Treatment vs. 0.003% CHX. ⁺p<0.05 Treatment vs. 0.001% CHX. ^{\hat{ }}p<0.05 vs. 0.003% HCX. ^{@}p<0.05 Treatment vs. 0.002% HCX. ^{%}p<0.05 Treatment vs. 0.001% HCX.

FIG. 14. Antimicrobial activity of different concentrations of 9-Hydroxycalabaxanthone (HCX). (A) S. mutans growth rate cultured for 5 h with different concentrations of chlorhexidine (CHX) and 9-Hydroxycalabaxanthone (HCX). (C) S. mutans Live/Dead Ratio cultured for 5 h with different concentrations of CHX and HCX. Data represent the mean±SEM. The negative control (C−) was bacterial suspension without any treatment. Results were statistically compared by ANOVA and Bonferroni as post hoc: *p<0.05 Treatment vs. C−. ^{#}p<0.05 Treatment vs. 0.01% CHX. ^{&}p<0.05 Treatment vs. 0.005% CHX. ^{$}p<0.05 Treatment vs. 0.003% CHX. ⁺p<0.05 Treatment vs. 0.001% CHX. ^{\hat{ }}p<0.05 vs. 0.003% HCX. ^{@}p<0.05 Treatment vs. 0.002% HCX. ^{%}p<0.05 Treatment vs. 0.001% HCX.

FIG. 15. Antimicrobial activity of different concentrations of HCX. P. aeruginosa growth rate cultured at 3 h with different concentrations of chlorhexidine (CHX) and 9-Hydroxycalabaxanthone (HCX). Data represent the mean±SEM. The negative control (C−) was bacterial suspension without any treatment. Results were statistically compared by ANOVA and Bonferroni as post hoc: *p<0.05 Treatment vs. C−. ^{#}p<0.05 Treatment vs. 0.01% CHX. ^{&}p<0.05 Treatment vs. 0.005% CHX. ^{$}p<0.05 Treatment vs. 0.003% CHX. ⁺p<0.05 Treatment vs. 0.001% CHX. ^{\hat{ }}p<0.05 vs. 0.003% HCX. ^{@}p<0.05 Treatment vs. 0.002% HCX. ^{%}p<0.05 Treatment vs. 0.001% HCX.

FIG. 16. Antifungal activity of different concentrations of HCX. C. albicans growth rate cultured for 24 h with different concentrations of chlorhexidine (CHX) and 9-Hydroxycalabaxanthone (HCX). Data represent the mean±SEM. The negative control (C−) was fungal suspension without any treatment. Results were statistically compared by ANOVA and Bonferroni as post hoc: *p<0.05 Treatment vs. C−. ^{#}p<0.05 Treatment vs. 0.01% CHX. ^{&}p<0.05 Treatment vs. 0.005% CHX. ^{$}p<0.05 Treatment vs. 0.003% CHX. ⁺p<0.05 Treatment vs. 0.001% CHX. ^{\hat{ }}p<0.05 vs. 0.003% HCX. ^{@}p<0.05 Treatment vs. 0.002% HCX. ^{%}p<0.05 Treatment vs. 0.001% HCX.

FIG. 17. Antimicrobial activity of different concentrations of HCX (A) S. pyogenes growth rate cultured for 24 h with different concentrations of chlorhexidine (CHX) and 9-Hydroxycalabaxanthone (HCX). (C) S. pyogenes Live/Dead Ratio cultured for 24 h with different concentrations of CHX and HCX. Data represent the mean±SEM. The negative control (C−) was bacterial suspension without any treatment. Results were statistically compared by ANOVA and Bonferroni as post hoc: *p<0.05 Treatment vs. C−. ^{#}p<0.05 Treatment vs. 0.01% CHX. ^{&}p<0.05 Treatment vs. 0.005% CHX. ^{$}p<0.05 Treatment vs. 0.003% CHX. ⁺p<0.05 Treatment vs. 0.001% CHX. ^{\hat{ }}p<0.05 vs. 0.003% HCX. ^{@}p<0.05 Treatment vs. 0.002% HCX. ^{%}p<0.05 Treatment vs. 0.001% HCX.

FIG. 18. Effect of different hydrogel formulations with and without 0.05% of 9-Hydroxycalabaxanthone (HCX) [Hyaluronic acid (HA), Gelatin Methacryloyl (GelMa), Alginate and Hydroxymethyl propyl cellulose (HPMC)] and commercial Periokin gel containing 0.2% of chlorhexidine (CHX) on the activity of LDH released into the medium. Data represent the mean±SEM (n=4). Results were statistically compared by Kruskal Wallis: *p<0.05 Treatment vs. C−. ^{#}p<0.05 Treatment vs. Commercial HA 0.2% CHX. ^{&}p<0.05 Treatment vs. HA. ^{$}p<0.05 Treatment vs. GelMA.

FIG. 19. Antimicrobial activity of different hydrogel formulations with and without 0.05% of 9-Hydroxycalabaxanthone (HCX) [Hyaluronic acid (HA), Gelatin Methacryloyl (GelMa), Alginate and Hydroxymethyl propyl cellulose (HPMC)] and commercial Periokin gel containing 0.2% of chlorhexidine (CHX). (A) S. epidermidis growth rate cultured for 3 h with different gels. Results are expressed as % vs C− that was set to 100%. (B) S. epidermidis Live/Dead Ratio cultured for 3 h with different gels. Data represent the mean±SEM (n=3). Results were statistically compared by ANOVA and Bonferroni as Post hoc: *p<0.05 Treatment vs. C−; ^{#}p<0.05 Treatment vs. Commercial HA 0.2% CHX; ^{&}p<0.05 Treatment vs. HA; ^{$}p<0.05 Treatment vs. GelMA; ⁺p<0.05 Treatment vs. HPMC; ^{@}p<0.05 Treatment vs. Alginate.

FIG. 20. Biocompatibility and antimicrobial test of periodontal gels. (A) MTT viability assay in HOE tissue after the application of periodontal gels. Results are expressed as % vs C− that was set to 100%. (B) MTT viability assay in HGE tissue after the application of periodontal gels. Results are expressed as % vs C− that was set to 100%. (C) P. gingivalis growth rate cultured for 10 h with different periodontal gels. Results are expressed as % vs C− that was set to 100%. (D) P. gingivalis Live/Dead Ratio cultured for 10 h with different periodontal gels. Data represent the mean±SEM (n=3). Results were statistically compared by ANOVA and Bonferroni as post hoc: *p<0.05 Treatment vs. C−; ^{#}p<0.05 Treatment vs. C+; ^{$}p<0.05 Treatment vs Periokin HA 0.02% CHX; ^{&}p<0.05 Treatment vs. Perio-Aid HA 0.02% CHX; ⁺p<0.05 Treatment vs. Bexident Chitosan 0.02% CHX; ^{@}p<0.05 Treatment vs. Mucorepair 0.02% Enoxolone.

FIG. 21. Biocompatibility and antimicrobial test of antiseptic skin gels. (A) MTT viability assay in RHE tissue after the application of antiseptic skin gels. Results are expressed as % vs C− that was set to 100%. (B) S. aureus growth rate cultured for 3 h with different antiseptic skin gels. (C) S. aureus Live/Dead Ratio cultured for 3 h with different antiseptic skin gels. Data represent the mean±SEM (n=3). Results were statistically compared by ANOVA and Bonferroni as post hoc: *p<0.05 Treatment vs. C−; ^{#}p<0.05 Treatment vs. Betadine gel; ^{$}p<0.05 Treatment vs. Cristalmina gel.

FIG. 22. Effect of 0.05% 9-hydroxycalabaxanthone (HCX) and 0.2% chlorhexidine (CHX) on the live/dead ratio (A) and CFU/mL (B) of bacterial plaque biofilm. Data represent the mean±SEM (n=6). Results were statistically compared by ANOVA and Bonferroni as post hoc for LIVE/DEAD and Kruskal-Wallis for CFU/mL: *p<0.05 Treatment vs. C−.

FIG. 23. Antimicrobial activity of different concentrations of HCX (A) C. acnes growth rate cultured for 24 h with different concentrations of chlorhexidine (CHX) and 9-Hydroxycalabaxanthone (HCX). Results are expressed as % vs C− that was set to 100%. (B) C. acnes Live/Dead Ratio cultured for 24 h with different concentrations of CHX and HCX. Data represent the mean±SEM (n=3). The negative control (C−) was bacterial suspension without any treatment. Results were statistically compared by ANOVA and Bonferroni as post hoc: *p<0.05 Treatment vs. C−.

FIG. 24. Evaluation of the antimicrobial activity of different concentrations of HCX (A), α-Mangostin—aMG (B), Norathryol—NRT (C), Formoxanthone A—FXA (D) and 1,3,7-trihydroxy-2-prenylxanthone—PX (E) on S. aureus and of the cytotoxicity thereof in gingival fibroblasts. In grey, the optimal concentration values for both parameters (S. aureus growth rate below 20% and Citotoxicity below 30%)

FIG. 25. Biocompatibility and antimicrobial test of periodontal gels formulated with HCX and aMG. (A) MTT viability assay in GTE tissue after the application of periodontal gels. Results are expressed as % vs C− that was set to 100%. (B) S. mutans growth rate cultured for 24 h with different periodontal gels. Results are expressed as % vs C− that was set to 100%. (D) S. mutans Live/Dead Ratio cultured for 24 h with different periodontal gels. Data represent the mean±SEM (n=3). Results were statistically compared by ANOVA and Bonferroni as post hoc: *p<0.05 Treatment vs. C−.

### DETAILED DESCRIPTION OF THE INVENTION

### Definitions

As used throughout, ranges are used as shorthand for describing each and every value that is within the range. Any value within the range can be selected as the terminus of the range. In addition, the compositions and the methods may comprise, consist essentially of, or consist of the elements described therein.

As used herein, “antimicrobial activity or properties” herein means activity as determined by any generally accepted in vitro or in vivo antibacterial assay or test.

An “oral surface” herein encompasses any soft or hard surface within the mouth including surfaces of the tongue, hard and soft palate, buccal mucosa, gums and dental surfaces.

A “dental surface” herein is a surface of a natural tooth or a hard surface of artificial dentition including a crown, cap, filling, bridge, denture, dental implant and the like.

As used in the present invention, it is noted that 9-hydroxycalabaxanthone may be in the form of salts, solvates or stereoisomers, preferably pharmaceutically acceptable salts, solvates or stereoisomers.

As already indicated, the present invention also provides “salts” of 9-hydroxycalabaxanthone. By way of illustration, said salts can be acid addition salts, base addition salts or metal salts, and can be synthesized from 9-hydroxycalabaxanthone by means of conventional chemical processes known by the person skilled in the art. See, generally, G. S. Paulekuhn, et al., “Trends in Active Pharmaceutical Ingredient Salt Selection based on Analysis of the Orange Book Database”, J. Med. Chem., 2007, 50: 6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Such salts are generally prepared, for example, by reacting the free acid or base forms of said compounds with a stoichiometric amount of the suitable base or acid in water or in an organic solvent or in a mixture of the two. Non-aqueous media such as ether, ethyl acetate, ethanol, acetone, isopropanol or acetonitrile are generally preferred. Illustrative examples of acid addition salts include inorganic acid addition salts such as, for example, hydrochloride, hydrobromide, hydroiodide, sulfate, pyrosulfate, bisulfate, sulfite, bisulfite, nitrate, phosphate, monohydrogen-phosphate, dihydrogenphosphate, meta-phosphate, pyrophosphate, etc., organic acid addition salts such as, for example, acetate, maleate, fumarate, citrate, oxalate, succinate, tartrate, malate, mandelate, methanesulfonate, p-toluenesulfonate, camphorsulfonate, propionates, decanoates, caprylates, acrylates, formates, isobutyrates, caproates, heptanoates, propiolates, malonates, succinates, suberates, sebacates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, lactates, y-hydroxybutyrates, glycolates, propanesulfonates, naphthalene-1-sulfonates, naphthalene-2-sulfonates, etc. Illustrative examples of base addition salts include inorganic base salts such as, for example, ammonium salts and organic base salts such as, for example, ethylenediamine, ethanolamine, N,N-dialkylenethanolamine, triethanolamine, glutamine, amino acid basic salts, etc. Illustrative examples of metal salts include, for example, sodium, potassium, calcium, magnesium, aluminum and lithium salts.

The term “solvate” according to this invention is to be understood as meaning any form of 9-hydroxycalabaxanthone which has another molecule (most likely a polar solvent) attached to it via non-covalent bonding. Examples of solvates include hydrates and alcoholates. Solvation methods are generally known in the state of the art.

As used herein, the term “stereoisomer” is a general term for all isomers of 9-hydroxycalabaxanthone that differ only in the orientation of their atoms in space. Therefore, stereoisomer compounds are molecules that are non-superimposable mirror images of each other and include enantiomers and diastereomers.

The terms “enantiomer” and “enantiomeric form” refer to one of the two stereoisomers of 9-hydroxycalabaxanthone that are mirror images of each other that are non-superimposable.

Enantiomer compounds are optically active, wherein one enantiomer rotates the plane of polarized light in one direction and the other one rotates the plane of polarized light in the opposite direction and form a racemic compound when present in equal quantities.

The term “racemic” or “racemate” refers to a mixture that has equal amounts of enantiomers and which mixture is optically inactive.

The terms “diastereomer” and “diastereomeric form” refer to stereoisomers of a compound with more than one chiral center that are not mirror images of one another.

The term “pharmaceutically acceptable” relates to molecular entities and compositions being physiologically tolerable and normally not causing an allergic reaction or similar adverse reaction, such as gastric discomfort, dizziness and the like, when they are administered to a human being. Preferably, as used in this description, the term “pharmaceutically acceptable” means approved by a governmental regulatory agency or listed in the US pharmacopoeia or another generally recognized pharmacopoeia for use in animals, and more particularly in humans.

The term “about” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Where particular values are described in the application and claims, unless otherwise stated the term “about” meaning within an acceptable error range for the particular value should be assumed. More preferably, the term “about” shall mean a range of up to 10% of a given value.

### DESCRIPTION OF EMBODIMENTS

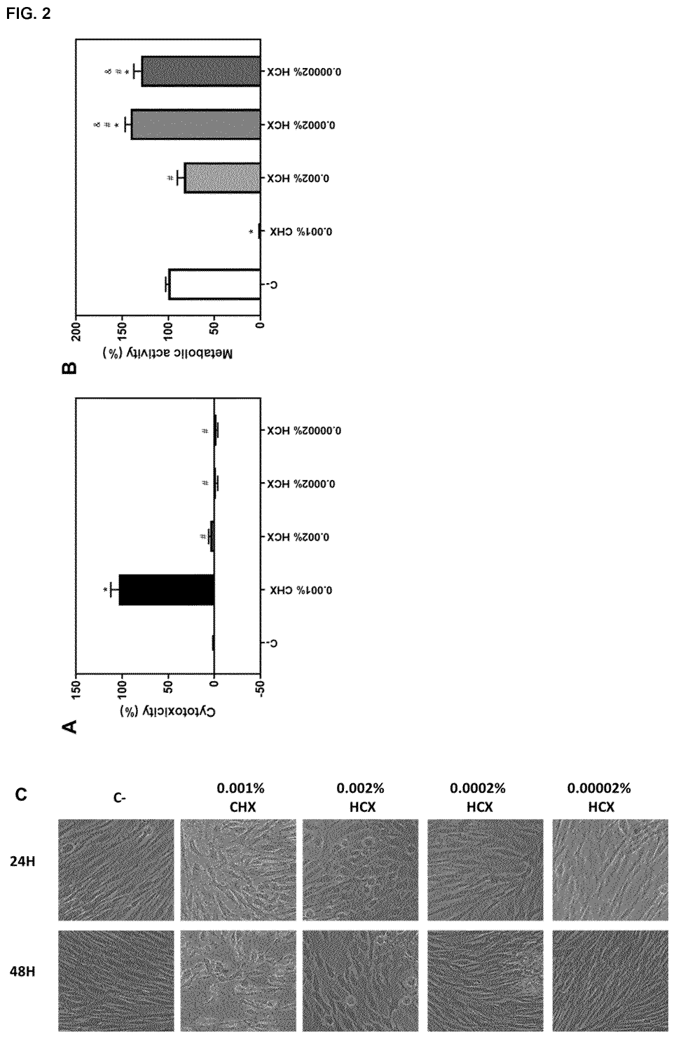

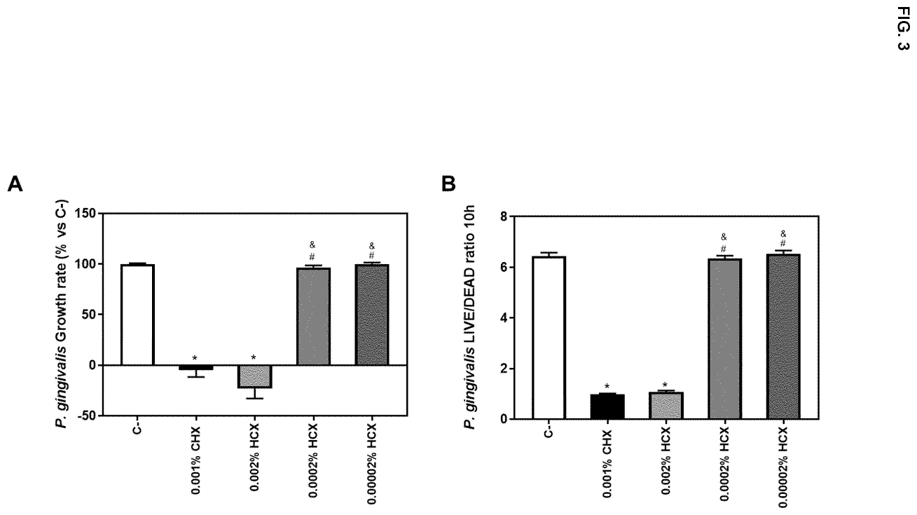

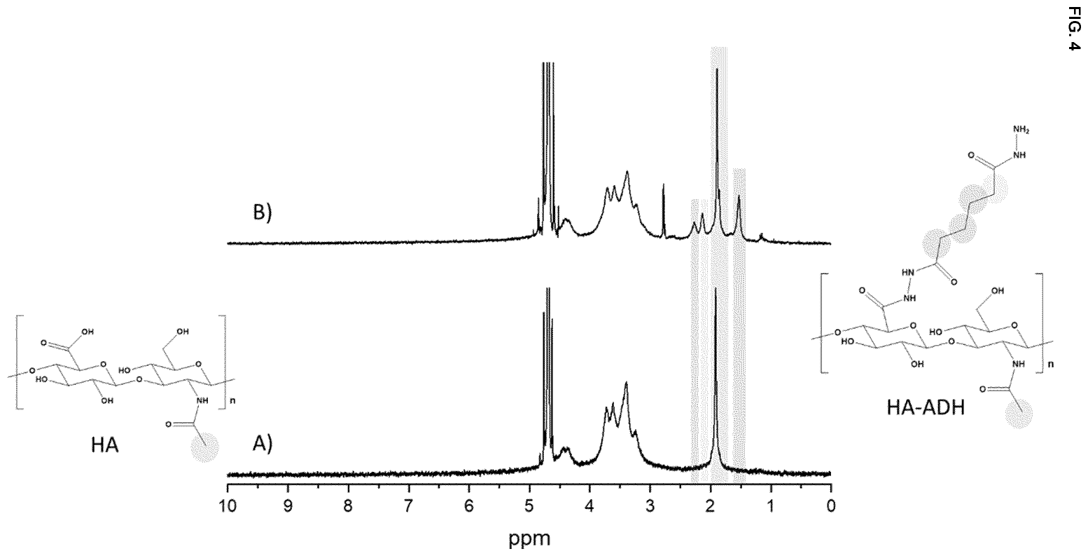

The present invention is confronted with the problem of providing an improved alternative antiseptic composition to chlorhexidine (CHX), that provides strong antimicrobial properties without cytotoxic effects. In this sense and as shown in example 1, in experiments with human gingival fibroblast cells, cytotoxicity of HCX evaluated by means of the activity of lactate dehydrogenase (LDH) released into the medium is not observed (FIG. 2). In addition, results of the evaluation of the antimicrobial properties of HCX with respect to P. gingivalis show that a concentration of 0.002% is sufficient to eliminate 100% of the bacteria (FIG. 3).

That is, HCX has antimicrobial properties without cytotoxic effects on human gingival cells.

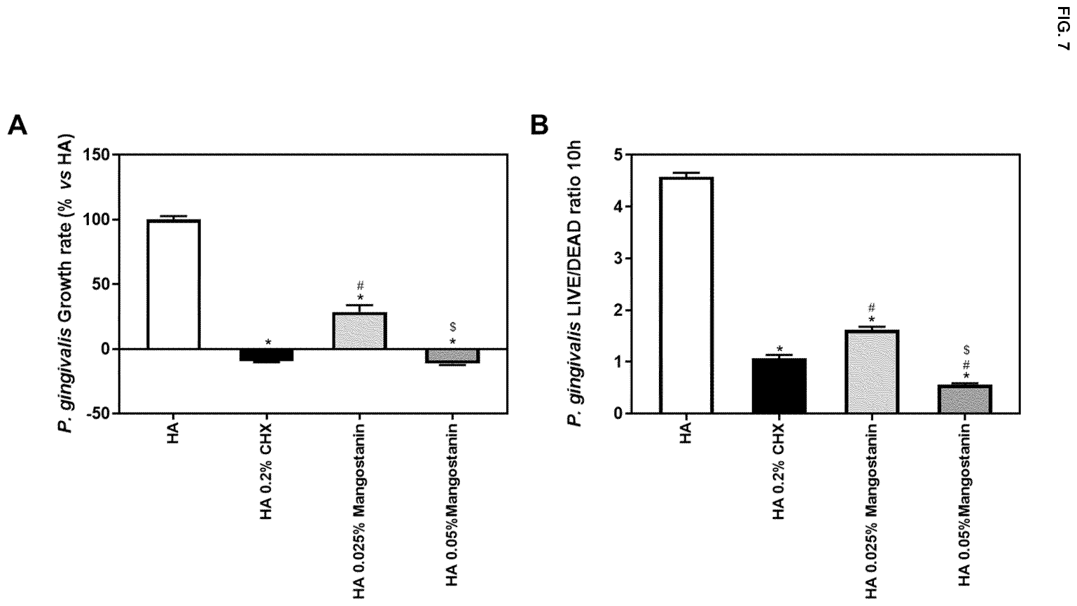

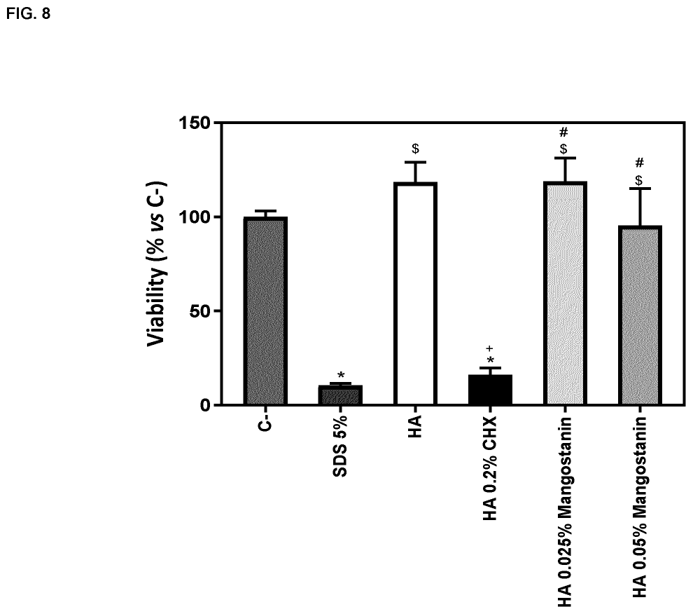

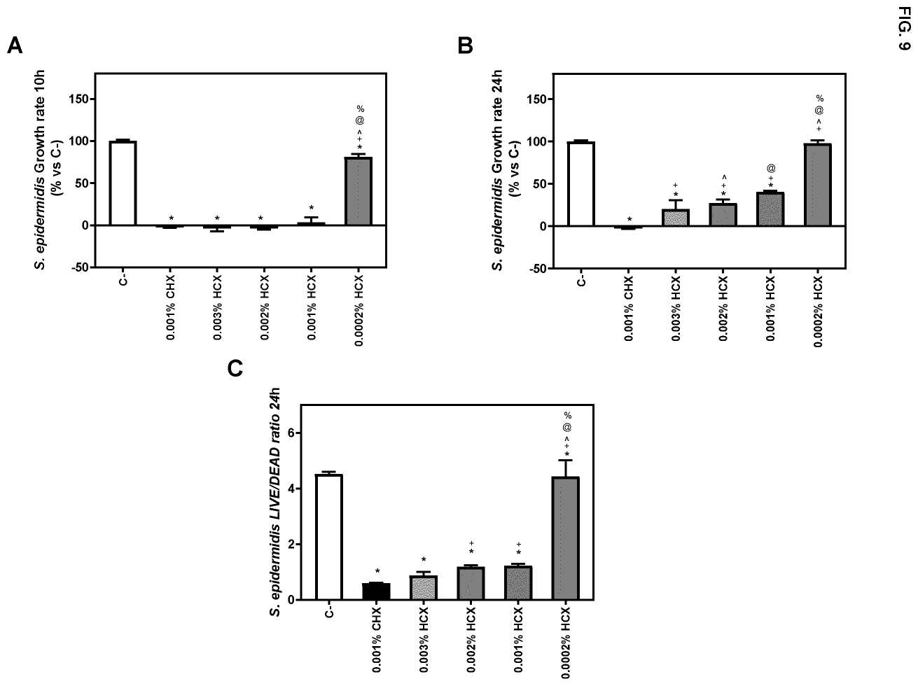

In addition, as shown in example 2, results of the formulation of the HA-HCX hydrogel show that the modified HA hydrogel absorbs large amounts of water (FIG. 6A) and that a controlled release of HCX into the aqueous medium occurs, maintaining a moderately sustained release of 20 μM after 10 h and up to 7 days (FIG. 6B). The quantification of released HCX shows that the concentration released and accumulated in one week is 120 μM. In addition, the results of FIG. 7 show that the gel modified with HCX has good antimicrobial properties, with a higher inhibition for a concentration of 0.05% and is similar to the effect produced by a commercial gel containing 0.2% chlorhexidine and unmodified hyaluronic acid (Periokin Hyaluronic 1%, Laboratorios Kin, Barcelona, Spain, HA 0.2% CHX). That is, again, HCX has good antimicrobial properties, moreover, solutions, hydrogels or gels containing HCX provide for a suitable antiseptic or antimicrobial composition for topical or oral administration in oral surfaces. Moreover, as shown in FIG. 8 said solutions, hydrogels or gels containing HCX present very good biocompatibility in a 3D gingival tissue, similar to modified HA without HCX and to an untreated gingival control tissue, for the two concentrations tested of 0.025% and 0.05%. This is in clear contrast to the commercial gel containing 0.2% chlorhexidine and unmodified hyaluronic acid (Periokin Hyaluronic 1%, Laboratorios Kin, Barcelona, Spain, HA 0.2% CHX) which presents toxic effects on the tissue.

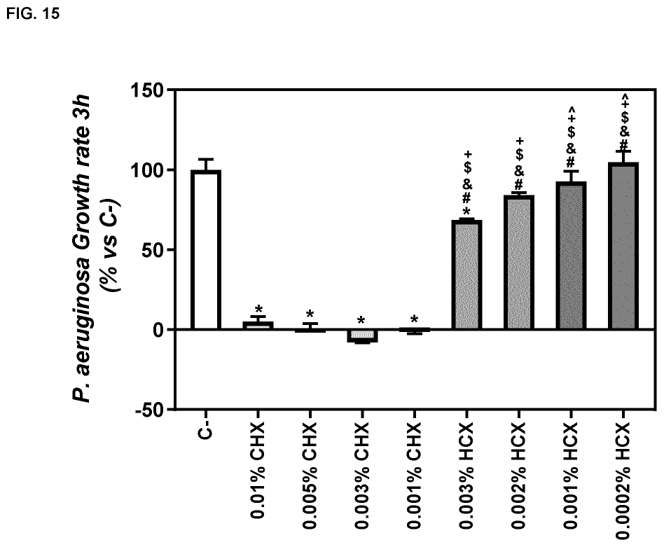

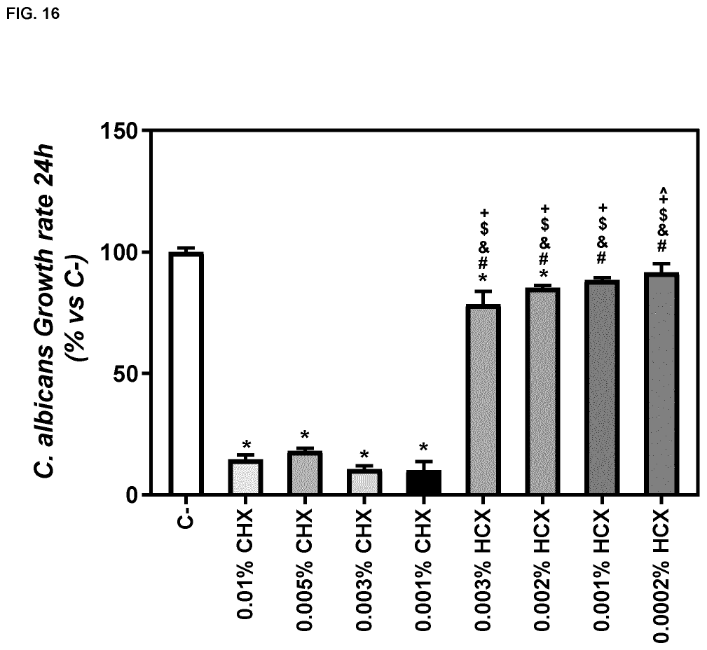

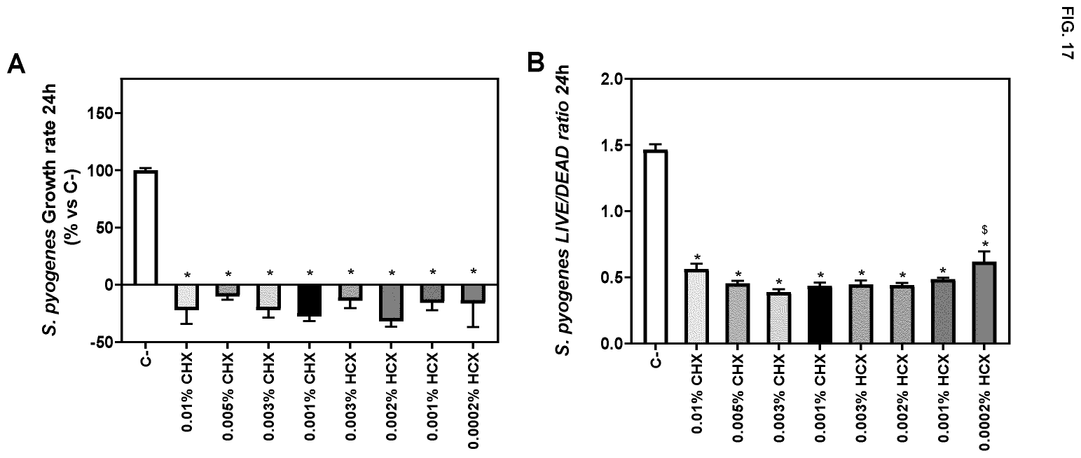

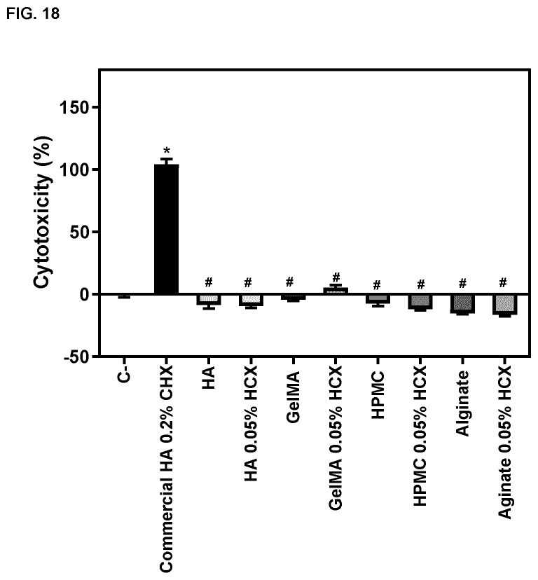

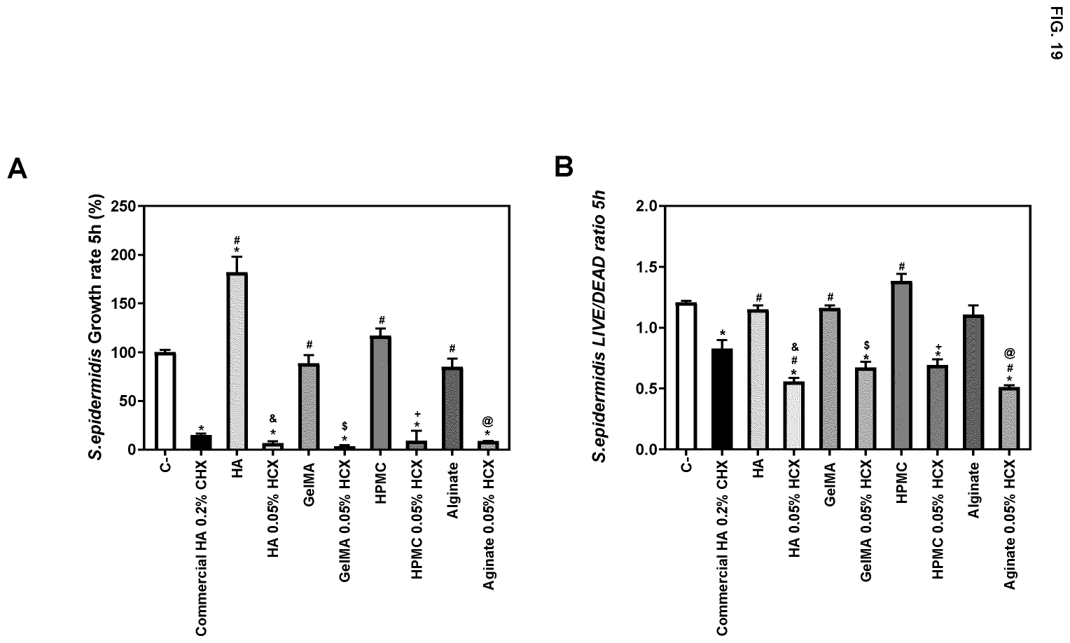

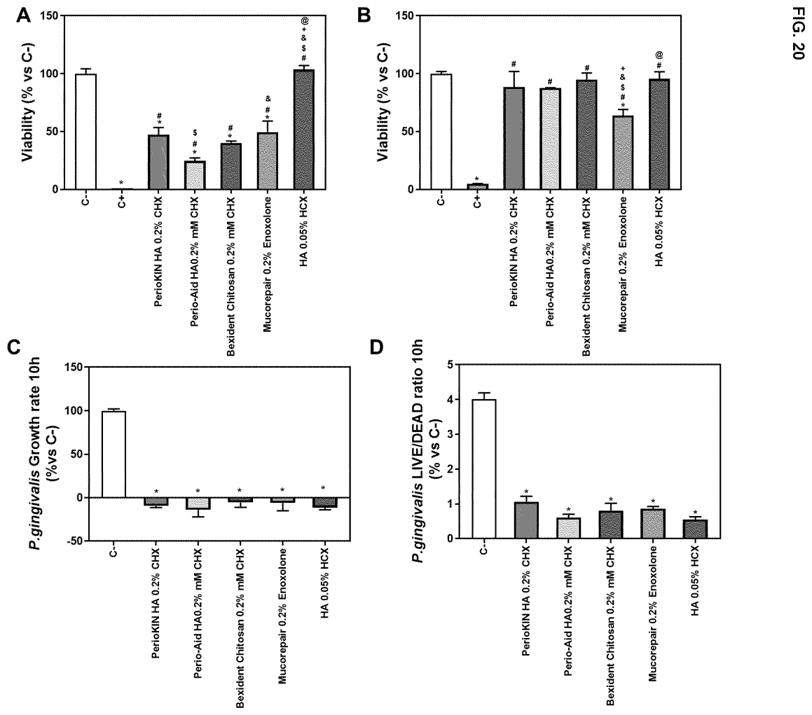

It is additionally noted that the results of the formulation of the HA-HCX hydrogel can be extended to other hydrogel formulations. In this sense, in example 12 we shall notice that individually, in experiments with human gingival fibroblast cells, the cytotoxicity evaluated by lactate dehydrogenase (LDH) activity released into the medium was not observed in the different hydrogels (HA, GelMa, Alginate and HPMC) tested therein formulated with and without HCX. On the contrary, the commercial Periokin gel containing 0.2% of CHX showed high cytotoxicity. The results of FIG. 15 show that all the different hydrogels formulated with 0.05% HCX (HA 0.05% HCX, GelMa 0.05% HCX, Alginate 0.05% HCX and HPMC 0.05% HCX) have good antimicrobial properties and are similar or better to the effect produced by a commercial gel containing 0.2% chlorhexidine.

Furthermore, example 5 clearly shows that a concentration of 0.001% of HCX incubated for 10 h on S. epidermidis is sufficient to eliminate 100% of bacteria and a concentration of 0.003% of HCX incubated for 24 h on S. epidermidis eliminates 80% of bacteria, which is similar to the effectivity of the most common antiseptic used for the skin, chlorhexidine at 0.001%.

Furthermore, for the particular case of periodontitis the results of example 6 show that the HA gel containing HCX can help to re-stabilize the oral microbiome, by increasing the presence of bacteria related to a healthy oral microbiota, and by reducing the present of microbiota associated with periodontitis.

Example 7 shows that that a concentration of 0.002% of HCX incubated for 3 h on S. aureus is sufficient to eliminate 100% of bacteria and a concentration of 0.001% of HCX incubated for 3 h on S. aureus eliminates 88% of bacteria, which is similar to the effectivity of the most common antiseptic used for the skin, such as chlorhexidine (CHX).

Example 8 shows that a concentration of 0.001% of HCX incubated for 5 h on S. mutans is sufficient to eliminate 90% of bacteria and a concentration of 0.0002% of HCX incubated for 5 h on S. mutans eliminates 70% of bacteria, which is similar to the effectivity of the most common antiseptic used for the oral cavity, chlorhexidine.

Examples 9 and 10 show that a concentration of 0.003% of HCX incubated for 3 h on P. aeruginosa eliminates 32% of bacteria and that a concentration of 0.003% of HCX incubated for 24 h on C. albicans eliminates 22% of fungal.

Example 11 shows that a concentration of 0.0002% of HCX incubated for 24 h on S. pyogenes is sufficient to eliminate 100% of bacteria, which is similar to the effectivity of the most common antiseptic used for the skin, such as chlorhexidine (CHX).

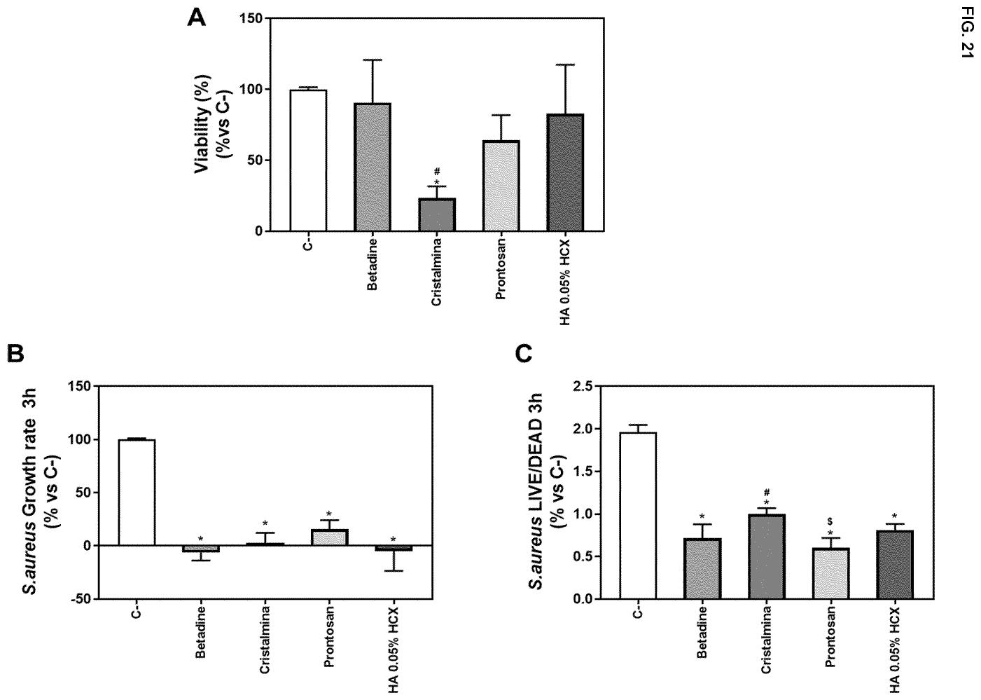

Example 13, in particular FIG. 20A, shows that the modified HA gel with 0.05% of HCX presents very good biocompatibility in a HOE tissue, similar to an untreated oral control tissue (C−). However, the commercial gels containing 0.2% chlorhexidine with unmodified hyaluronic acid or chitosan (Periokin HA 0.2% CHX, Perio-Aid HA 0.2% CHX and Bexident Chitosan 0.2% CHX), and the commercial gel containing 0.2% of Enoxolone (Mucorepair 0.2% Enoxolone) presented toxic effects on the HOE, below 50% of viability. Moreover, FIG. 20B shows that Periokin HA 0.2%, Perio-Aid HA 0.2% CHX, Bexident Chitosan 0.2% CHX and the modified HA gel with 0.05% of HCX presents very good biocompatibility in a HGE tissue, similar to an untreated oral control tissue (C−). However, the commercial gel Mucorepair 0.2% Enoxolone presented toxic effects on the GTE, below 64% of viability. In addition, the results of FIGS. 20C and D show that all periodontal gels, illustrated in example 13, have a high antibacterial activity against P. gingivalis, a characteristic bacterium of periodontal diseases.

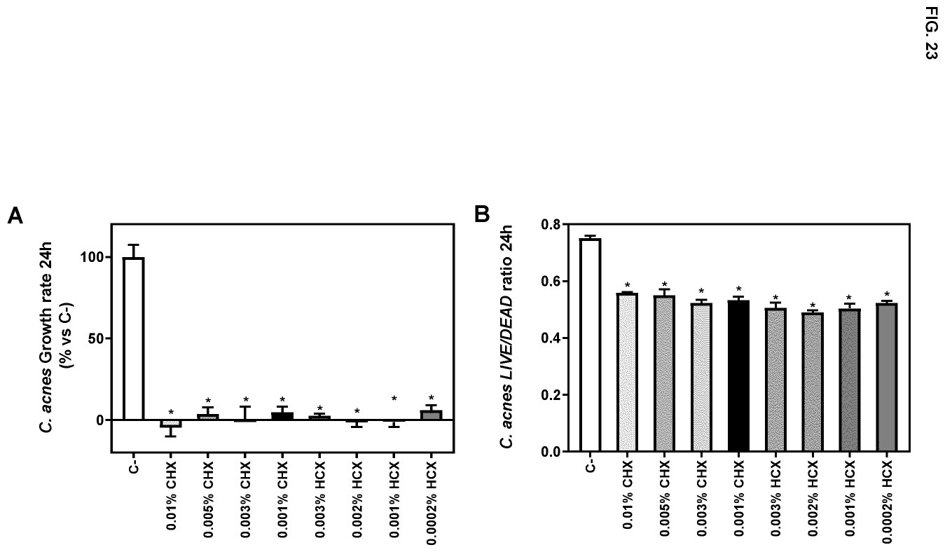

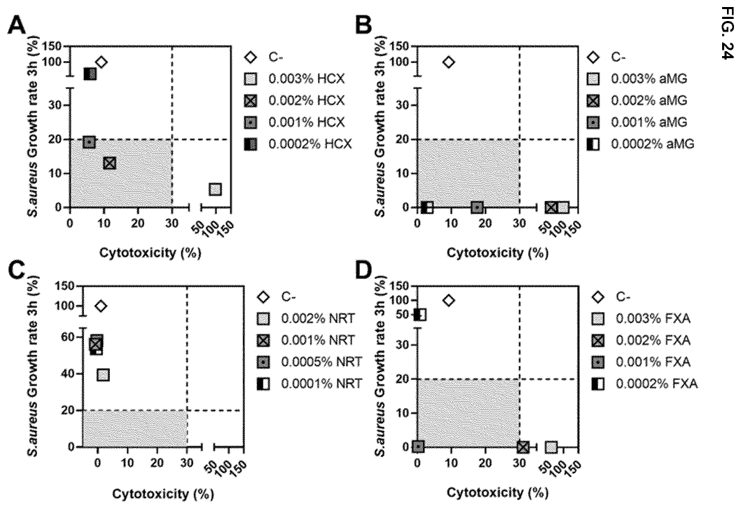

Lastly, FIG. 22 shows that the treatment with 0.05% HCX reduced plaque biofilm bacteria formation with results equal to 0.2% chlorhexidine, the positive control, and FIG. 23 shows that a concentration of 0.0002% of HCX incubated for 24 h on C. acnes is sufficient to eliminate 96% of bacteria, which is similar to the effectivity of the most common antiseptic used for the skin.

Therefore, the compositions of the present invention comprising 9-hydroxycalabaxanthone (HCX) inhibit the growth of various skin and oral bacteria that are implicated, for example, in forming plaque and causing oral diseases and skin diseases or skin infections in lesions such as burns or chronic wounds. Moreover, although 9-hydroxycalabaxanthone (HCX) is specially preferred, the present invention is not limited to compositions comprising HCX as the compositions of the present invention are also envisaged to comprise or consists of the specific xanthone or derivatives thereof disclosed herein (see formula I below).

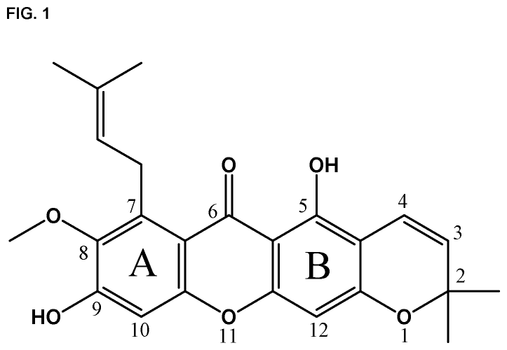

In this sense, in a general sense, xanthones are secondary metabolites found in some higher plants, fungi, and lichens. The xanthone skeleton (the word “xanthon” is derived from the Greek word xanthos, meaning yellow) is a planar, conjugated ring system composed of carbon 14 (aromatic ring A) and carbon 58 (aromatic ring B), combined through a carbonyl group and an oxygen atom (see FIG. 1 or the ring structure below). The simplest member of the class, 9H-xanthen-9-one, is a symmetrical compound with a dibenzo-γ-pyrone skeleton. The numbering starts from ring A, while ring B is given prime locants or consecutively numbered from ring A.

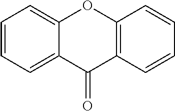 [SMILES: O=c1c2ccccc2oc2ccccc12]

Xanthones are classified as oxygenated xanthones, prenylated xanthones, xanthone glycosides, xanthonolignoids, bis-xanthones, and miscellaneous xanthones, which entail caged xanthones. The polyphenolic xanthones are also divided into subclasses depending upon the degree of oxygenation. These subgroups entail non-, mono-, di-, tri-, tetra-, penta-, and hexa-oxygenated substances. The other xanthone subclasses rely more upon the level of oxidation of ring A, which can occur either as fully aromatic or as dihydro-, tetrahydro-, and hexahydro derivatives, or in monomeric or dimeric form. Several xanthones could be present as hydroxylated xanthones with prenyl or geranyl units. Prenylated xanthones are mainly present in the Clusiaceae family, while the Gentianaceae family has oxygenated xanthones.

Consequently, in a first aspect, the present invention, refers to an antiseptic composition comprising a xanthone as shown in formula I below, preferably having one or more hydroxyl groups, one or more prenyl groups for targeting bacteria through its interaction with cell membranes, and preferably also having one or more methoxy groups to enhance its interaction with cell membranes. With respect to xanthones of formula I below, hydroxylation is preferred in positions R1, R3 and R6, but also in decreasing preference in position R7, then R5 and R8. Prenylation is preferred in positions R2 and R8, which can be or not cycled, but also prenylation can be present in decreasing preference in positions R4, then R5 and last R7. Methoxy groups are preferred in position R7, but also in R3, then R6 and R5.

Specifically, the present invention refers to a composition, preferably an antiseptic composition, comprising a compound having the structure of formula I below:

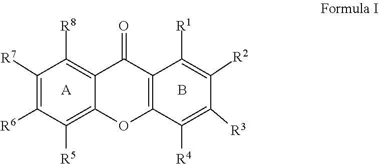

wherein
R1 can be a hydroxyl group (−OH) or a hydrogen atom (−H);
R2 can be a prenyl group or a hydrogen atom, wherein when R2 is a prenyl group and R3 or R1 are a hydroxyl group or a methoxy group, R2 and R3 or R2 and R1 can optionally link together forming a ring;
R3 can be a hydroxyl group, a methoxy group or a hydrogen atom;
R4 can be a prenyl group or a hydrogen atom;
R5 can be a hydroxyl group, a prenyl group, a methoxy group or a hydrogen atom;
R6 can be a hydroxyl group, a methoxy group or a hydrogen atom;
R7 can be a hydroxyl group, a prenyl group, a methoxy group or a hydrogen atom; and
R8 can be a prenyl group or a hydrogen atom, wherein when R8 is a prenyl group and R7 is a hydroxyl group or a methoxy group, R8 and R7 can optionally link together forming a ring;
or any salts, solvates, stereoisomers or enantiomers thereof (from hereinafter a composition comprising such Formula I compound shall be refer to herein as the composition or antiseptic composition of the present invention including any salts, solvates, stereoisomers or enantiomers thereof). In addition, from hereinafter any reference to a xanthone or xanthone compound according to the invention refers to any xanthone compound or molecule encompass under formula I above including any salts, solvates, stereoisomers or enantiomers thereof.

As used herein, a “prenyl group” shall be understood as the following chemical structure:

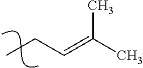

As used herein, a “methoxy group” shall be understood as the functional group consisting of a methyl group bound to oxygen (—O—CH₃)

Preferably, the term “antiseptic composition” is preferably, although not limited, understood as a composition including among others an antiseptic agent that reduces (bacterirostatic) or stops (bactericidal) the growth of potentially harmful microorganisms on the skin (compromised or not), such as but not restricted to Staphylococcus epidermidis, Staphylococcus aureus, Cutibacterium acnes, Streptococcus piogenes, or Epidermolysis bullosa, or on the oral mucosa and gingiva, such as Porphyromonas gingivalis, Aggregatibacter actinomycetemcomitans, Tannerella forsythia, Treponema denticola, Fusobacterium nucleatum, Streptococcus mutans.

Preferably, as used in the present invention, when referring to the skin, oral mucosa, oral cavity, gingiva etc. . . . , it is understood that this reference is made to that part of the body of a mammal, preferably of a human subject.

In a preferred embodiment, the compound having the structure of formula I is further characterized by
R1, R3 and R6 being independently selected from a hydroxyl group or a hydrogen atom;
R2 and R8 being independently selected from a prenyl group or a hydrogen atom; wherein when R2 is a prenyl group and R3 or R1 are a hydroxyl group or a methoxy group, R₂ and R₃ or R2 and R1 can optionally link together forming a ring; and wherein when R8 is a prenyl group and R7 is a hydroxyl group or a methoxy group, R₈ and R₇ can optionally link together forming a ring; and
R7 and R5 being independently selected from a methoxy group or a hydrogen atom;
R4 being preferably a hydrogen atom:

Including any salts, solvates, stereoisomers or enantiomers thereof.

Preferably, the xanthone or xanthone compound according to the invention is 9-hydroxycalabaxanthone (HCX) of formula II below, or any salts, solvates, stereoisomers or enantiomers thereof.

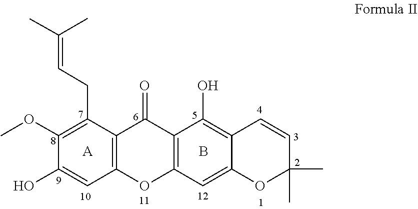

A modified formula II is also encompassed in the present invention as a 9-hydroxycalabaxanthone without the prenyl cyclization at position R2, that is a formula I compound wherein R1 is a hydroxyl group, R2 is a prenyl group, R3 is a hydroxyl group, R4 and R5 are hydrogen atoms, R6 is a hydroxyl group, R7 is a methoxy group and R8 is a prenyl group.

Preferably, the antiseptic composition of the present invention is for use as a topical or oral antiseptic agent in a subject in need thereof. It is noted that the amount of xanthone or xanthone compound according to the invention employed in the antiseptic composition of the present invention will depend upon the nature of the product (mouthwashes, gels, toothpastes, sprays, for example) and the condition/bacteria to be treated. However, preferably, the xanthone or xanthone compound according to the invention shall be used at a concentration between 0.1 μM and 100 mM.

At any rate, in case the antiseptic composition of the present invention is in the form of a gel or hydrogel, preferably those applicable or that are applied to and left on the skin, for the treatment of diseases such as acne, impetigo, psoriasis, erysipelas, necrotizing fasciitis, cellulitis, folliculitis, furuncles and carbuncles, scalded skin syndrome, etc. it is preferred that the xanthone or xanthone compound according to the invention be present in the antiseptic composition of the present invention in an amount of about 0.2% to about 0.0002% by weight/volume of the total composition, preferably about 0.05% to about 0.005% by weight/volume of the total composition.

In the context of the present invention a gel or hydrogel applicable to the skin shall be understood as a composition prepared by diluting the required amount of the xanthone to achieve the desired concentration in milliQ H₂O. Then, hyaluronic acid, or other gel or hydrogel, are dissolved in the aqueous solution with [HCX] or related xanthones to form the gel.

On the other hand, in case the antiseptic composition of the present invention is in the form of an antiseptic solution for skin disinfection or hair follicle treatment (folliculitis, furuncles and carbuncles), it is preferred that the xanthone or xanthone compound according to the invention be present in the said antiseptic solution in an amount of about 0.00001 to about 0.01% by weight/volume, preferably about 0.0002 to 0.002% by weight/volume of the total composition.

In the context of the present invention an “antiseptic solution for skin disinfection or hair follicle treatment” shall be understood as a composition prepared by diluting the xanthone at the right concentration in the desired final antiseptic solution composition.

Furthermore, in case the antiseptic composition of the present invention is in the form of an antibacterial oral hygiene composition in aqueous form (such as mouthwashes and solutions for halitosis), it is preferred that the xanthone or xanthone compound according to the invention be present in said compositions in an amount of about 0.0001% w/v to about 0.1% w/v of the total composition.

In the context of the present invention an “antibacterial oral hygiene composition in aqueous form” shall be understood as a composition prepared by diluting the xanthone at the right concentration in the desired final oral hygiene solution composition.

In case the antiseptic composition of the present invention is in the form of antibacterial gels to be used in the oral cavity as periodontal gels, it is preferred that a xanthone or xanthone compound according to the invention be present in said compositions in an amount of about 0.2% w/v to about 0.0002% w/v, preferably about 0.05% to about 0.005% w/v of the total composition.

In the context of the present invention “antibacterial gels” shall be understood as a composition prepared by diluting the required amount of the xanthone to achieve the desired concentration in milliQ H₂O. Then, hyaluronic acid, or other gel or hydrogel, are dissolved in the same aqueous solution with [HCX] or related xanthones to form the gel. It is herein noted that in case the antiseptic composition of the present invention is in the form of a gel or hydrogel, said composition may comprise any of the values (in % w/v of the total composition) indicated throughout the description including those indicated in the claims or examples.

It is herein further noted that in case the antiseptic composition of the present invention is the form of an aqueous composition or solution, said composition may comprise any of the values (in % w/v of the total composition) indicated throughout the description including those indicated in the claims or examples.

Notwithstanding the above, the composition may alternatively comprise between 1 μM and 5 mM or 3 mM, preferably between 1 μM, 10 μM, 25 μM, or 50 μM and 0.5 mM, more preferably between 1 μM, 10 μM, 25 μM, or 50 μM and 0.2 mM, more preferably between 1 μM, 10 μM, 25 μM, or 50 μM and 0.15 mM, more preferably between 50 μM and about 0.1 mM, of the xanthone or xanthone compound according to the invention, preferably 9-hydroxycalabaxanthone or salts, solvates, stereoisomers or enantiomers thereof (HCX), and is for use as a topical antiseptic agent. Also preferably, the composition comprises between 1 μM and 5 mM or 3 mM, preferably between 1 μM, 10 μM, 25 μM, or 50 μM and 0.5 mM, more preferably between 1 μM, 10 μM, 25 μM, or 50 μM and 0.2 mM, more preferably between 1 μM, 10 μM, 25 μM, or 50 μM and 0.15 mM, more preferably between 50 μM and about 0.1 mM, of the xanthone or xanthone compound according to the invention, preferably 9-hydroxycalabaxanthone or salts, solvates, stereoisomers or enantiomers thereof (HCX), and is for use as an oral antiseptic agent. It is noted that when the xanthone or xanthone compound according to the invention, preferably 9-hydroxycalabaxanthone or salts, solvates, stereoisomers or enantiomers thereof (HCX), is formulated as a hydrogel, concentrations of preferably up to 3 mM of this compound is included in the hydrogel as these types of formulations provide for a slow-release profile. For other oral formulations, concentrations of between 50 μM and 0.1 mM are specially preferred.

In a preferred embodiment, the antiseptic composition of the present invention is for use in the prevention or treatment, via topical administration, of diseases of microbial origin in the skin such as those caused by Staphylococcus aureus, Staphylococcus epidermidis Cutibacterium acnes, Streptococcus pyogenes, Pseudomonas aeruginosa, Candida albicans and/or Epidermolysis bullosa, preferably at a concentration between 1 μM and 5 mM or 3 mM, preferably between 1 μM, 10 μM, 25 μM, or 50 μM and 0.5 mM, more preferably between 1 μM, 10 μM, 25 μM, or 50 μM and 0.2 mM, more preferably between 1 μM, 10 μM, 25 μM, or 50 μM and 0.15 mM, more preferably between 50 μM and about 0.2 mM or 0.1 mM. It is at any rate noted that, as already stated, in the case of products like gels or hydrogels that are applied to and left on the skin, for the treatment of diseases such as acne, impetigo, psoriasis, erysipelas, necrotizing fasciitis, cellulitis, folliculitis, furuncles and carbuncles, scalded skin syndrome, etc. it is preferred that the xanthone or xanthone compound according to the invention be present in the antiseptic composition of the present invention in an amount of about 0.2% to about 0.0002% by weight/volume of the total composition, preferably about 0.05% to about 0.005% by weight/volume of the total composition. On the other hand, in the case of antiseptic solutions for skin disinfection or hair follicle treatment (folliculitis, furuncles and carbuncles), it is preferred that the xanthone or xanthone compound according to the invention be present in the antiseptic composition of the present invention in an amount of about 0.00001 to about 0.01% by weight/volume, preferably about 0.0002 to 0.002% by weight/volume of the total composition.

In another preferred embodiment, the antiseptic composition of the present invention is for use in oral hygiene and/or for the prevention or treatment, via topical administration, of oral diseases of microbial origin such as those selected from the list consisting of periodontal diseases, plaque, caries, halitosis, gingivitis, mucositis, mycosis, and oral aphthous ulcers. Preferably, the disease is periodontitis and the related microbes are Porphyromonas gingivalis, Aggregatibacter actinomycetemcomitans, Tannerella forsythia, Treponema denticola, Fusobacterium nucleatum, Streptococcus mutans, preferably at a concentration between 1 μM and 5 mM or 3 mM, preferably between 1 μM, 10 μM, 25 μM, or 50 μM and 3 mM, preferably between 1 μM, 10 μM, 25 μM, or 50 μM and 2 mM, preferably between 1 μM, 10 μM, 25 μM, or 50 μM and 0.5 mM, more preferably between 1 μM, 10 μM, 25 μM, or 50 μM and 0.2 mM, more preferably between 1 μM, 10 μM, 25 μM, or 50 μM and 0.15 mM, more preferably between 50 μM and about 3 mM. It is at any rate noted that in the case of an antibacterial oral hygiene compositions in aqueous form (such as mouthwashes and solutions for halitosis), it is preferred that the xanthone or xanthone compound according to the invention be present in said compositions in an amount of about 0.0001% w/v to about 0.1% w/v of the total composition. In the case of antibacterial gels to be use in the oral cavity it is preferred that a xanthone or xanthone compound according to the invention be present in said compositions in an amount of about 0.2% w/v to about 0.0002% w/v, preferably about 0.05% to about 0.005% w/v of the total composition. The antiseptic composition of the present invention may be thus topically applied to the skin, or to mucosae or to oral surfaces in the oral cavity. The antiseptic composition of the present invention promotes overall health, in particular oral health including preventing plaque formation, caries formation, calculus formation, halitosis, gingivitis, and periodontitis, among others. For example, as shown herein, in an embodiment of the present invention, where an oral care composition comprises an orally acceptable delivery-carrier, such as hyaluronic acid, and a safe and effective amount of HCX, the antibacterial activity is highly efficacious against both gram-positive and gram-negative bacteria.

As noted above, the antiseptic composition of the present invention comprises at least a xanthone or xanthone compound according to the invention, preferably 9-hydroxycalabaxanthone or salts, solvates, stereoisomers or enantiomers thereof (HCX), preferably at a concentration between 0.1 μM and 100 mM, more preferably at any of the concentrations indicated above depending on the formulation type and/or the conditioned to be treated. Alternatively, the antiseptic composition of the present invention may comprise or consists of a sole active ingredient or component, a xanthone or xanthone compound according to the invention, preferably 9-hydroxycalabaxanthone or salts, solvates, stereoisomers or enantiomers thereof (HCX), preferably at a concentration between 0.1 μM and 100 mM, more preferably at any of the concentrations indicated above depending on the formulation type and/or the conditioned to be treated. The fact that the composition may consist of a sole active ingredient does not preclude the possibility of other components or ingredients being present such as excipients, adjuvants etc. . . . . Moreover, the composition may consist of more than a sole active ingredient.

As it is understood in the art, the amount to be included of the xanthone or xanthone compound according to the invention, preferably 9-hydroxycalabaxanthone or salts, solvates, stereoisomers or enantiomers thereof (HCX), in the antiseptic composition of the present invention should be safe and effective e.g., microbial growth, at the target site of a host to be treated, without undue adverse side effects (such as toxicity, invitation, or allergic response), commensurate with a reasonable benefit/risk ratio when used in the manner of this invention. The specific “safe and effective amount” will vary with such factors as the particular condition being treated, the physical condition of the patient, the duration of treatment, the nature of concurrent therapy (if any), the specific dosage form to be used, the excipient employed, the solubility of the active ingredient therein, and the dosage regimen desired for the oral composition. At any rate, the composition preferably comprises between 1 μM and 5 mM or 3 mM, preferably between 1 μM, 10 μM, 25 μM, or 50 μM and 0.5 mM, more preferably between 1 μM, 10 μM, 25 μM, or 50 μM and 0.2 mM, more preferably between 1 μM, 10 μM, 25 μM, or 50 μM and 0.15 mM, more preferably between 50 μM and about 0.2 mM or 0.1 mM, of a xanthone or xanthone compound according to the invention, preferably 9-hydroxycalabaxanthone or salts, solvates, stereoisomers or enantiomers thereof (HCX), when used as topical antiseptic agent (directly on the skin or mucosae); preferably in the case of products like gels or hydrogels that are applied to and left on the skin, for the treatment of diseases such as acne, impetigo, psoriasis, erysipelas, necrotizing fasciitis, cellulitis, folliculitis, furuncles and carbuncles, scalded skin syndrome, etc. it is preferred that the xanthone or xanthone compound according to the invention be present in the antiseptic composition of the present invention in an amount of about 0.2% to about 0.0002% by weight/volume, preferably about 0.05% to about 0.005% by weight/volume of the total composition. On the other hand, in the case of antiseptic solutions for skin disinfection or hair follicle treatment (folliculitis, furuncles and carbuncles), it is preferred that the xanthone or xanthone compound according to the invention be present in the antiseptic composition of the present invention in an amount of about 0.00001 to about 0.01% by weight/volume, preferably about 0.0002 to 0.002% by weight/volume of the total composition.

On the other hand, the composition preferably comprises between 1 μM and 5 mM or 3 mM, preferably between 1 μM, 10 μM, 25 μM, or 50 μM and 3 mM, preferably between 1 μM, 10 μM, 25 μM, or 50 μM and 2 mM, preferably between 1 μM, 10 μM, 25 μM, or 50 μM and 0.5 mM, more preferably between 1 μM, 10 μM, 25 μM, or 50 μM and 0.2 mM, more preferably between 1 μM, 10 μM, 25 μM, or 50 μM and 0.15 mM, more preferably between 50 μM and about 3 mM, of a xanthone or xanthone compound according to the invention, preferably 9-hydroxycalabaxanthone or salts, solvates, stereoisomers or enantiomers thereof (HCX), when used as an oral antiseptic agent in the oral surface. Preferably, in the case of an antibacterial oral hygiene composition in aqueous form (such as mouthwashes and solutions for halitosis), it is preferred that the xanthone or xanthone compound according to the invention be present in said compositions in an amount of about 0.0001% w/v to about 0.1% w/v of the total composition. In the case of antibacterial gels to be use as periodontal gels or aft as it is preferred that a xanthone or xanthone compound according to the invention be present in said compositions in an amount of about 0.2% w/v to about 0.0002% w/v, preferably about 0.05% to about 0.005% w/v of the total composition.

Please thus note that depending on the formulation of the oral care product, the concentration shall vary.

As indicated above, in various embodiments, the antiseptic composition of the present invention is used to prepare oral or topical compositions such as, hydrogels, dentifrices, gels, creams, and mouth rinses. Preferably, the antiseptic composition of the present invention is used to prepare oral compositions in the form of a hydrogel, mouthwash, gel, toothpaste or other forms of oral or topical use.

Preferably, the antiseptic composition of the present invention is a cosmetic or pharmaceutical composition containing a xanthone or xanthone compound according to the invention, preferably 9-hydroxycalabaxanthone or salts, solvates, stereoisomers or enantiomers thereof (HCX), in the form of gel or hydrogel, aqueous solution, soap, or other presentations, which can be used efficiently in the antiseptic treatment of the buccal cavity, as well as in the disinfection of surfaces such as body surfaces, for example, in the treatment of medical conditions associated with infection (such as acute or chronic wounds, burns, and surgical wounds), or disinfection of the skin before a surgical procedure. As already indicated through-out the specification, the advantage of using HCX is that it has potent antimicrobial effects, in the absence of toxicity in living tissues, such as the skin (both healthy and compromised) such as mucosae, including oral mucosa and gums.

In various embodiments, the antiseptic composition of the present invention, at any of the concentrations above established, is a topical or oral care composition. However, additional antibacterial, antioxidant and/or anti inflammatory active ingredients may be included in the oral care compositions. If added, the antibacterial, active ingredients should not react with or detract from the efficacy and bioavailability of the xanthone or xanthone compound according to the invention, preferably 9-hydroxycalabaxanthone or salts, solvates, stereoisomers or enantiomers thereof (HCX). Suitable antibacterial, anti-inflammatory, and/or antioxidant agents for use in addition to the 9-hydroxycalabaxanthone (HCX) or salts, solvates, stereoisomers or enantiomers thereof, include any known in the art, botanical or synthetic, vitamins, proteinoid agents, peptides, minerals, salts and/or biologics.

Other natural extracts that are known antimicrobial agents are those listed in the International Cosmetic Ingredient Dictionary and Handbook, Tenth Bd., 2004.

In various embodiments, the additional agents added to the oral composition of the present invention comprise from 0.0001% to 10%, preferably from 0.001% to 5%, more preferably from 0.01% to 3%, depending on the concentration of the active compounds and form of the oral composition.

In certain embodiments, the oral or topical compositions of the present invention optionally comprise one or more additional active ingredients. Such material may be operable for the prevention or treatment of a condition or disorder of hard or soft tissue of the oral cavity or of the mucosae or skin, for the prevention or treatment of a physiological, localized or systemic disorder or condition, or to provide a cosmetic benefit. Optional oral or topical care actives among those useful herein include antibacterial agents, antiplaque agents, anti-adhesion, antioxidant, anticaries agents, anti-inflammatory agents, desensitizing agents, whitening agents, tartar control agents, periodontal actives, nutrients, abrasives, breath freshening agents, malodour control agents, tooth desensitizers, salivary stimulants, and combinations thereof, such as those known to one of skill in the art.

Various optional oral care actives may be included in the oral composition of the present invention including those described above, such as antibacterial agents, antiplaque agents, anti-adhesion (that prevent adhesion of plaque to an enamel surface), antioxidant (such as Vitamin E or coenzyme Q10), anticaries agents, desensitizing agents (such as potassium citrate, potassium tartrate, potassium chloride, potassium sulfate and potassium nitrate), whitening agents (such as, urea peroxide, sodium percarbonate, sodium perborate and polyvinylpyrrolidone-H202); compatible enzymes; anti-inflammatory agents (such as, steroidal agents including flucinolone and hydrocortisone, and nonsteroidal agents (NSAIDs)), tartar control agents, periodontal actives, chlorophyll compounds, nutrients (such as vitamins, minerals, and amino acids, lipotropics, fish oil, coenzymes and the like) abrasives, breath freshening/malodour control agents (such as zinc salts such as zinc gluconate, zinc citrate, zinc chlorite, and α-ionone), and salivary stimulants (such as such as citric, lactic, malic, succinic, ascorbic, adipic, fumaric and tartaric acids); and any other suitable ingredients for oral care known to one of skill in the art.

In various embodiments, the oral compositions of the present invention comprise antitartar agents to prevent and/or minimize calculus formation. One or more of such agents can be present.

Suitable anticalculus agents include without limitation: phosphates and polyphosphates. Phosphate and polyphosphate salts are generally employed in the form of their wholly or partially neutralized water-soluble cationic species (e.g., potassium, sodium or ammonium salts, and any mixtures thereof). Thus, useful inorganic phosphate and polyphosphate salts illustratively include monovalent cations with monobasic, dibasic and tribasic phosphates; tripolyphosphate and tetrapolyphosphate; mono-, di-, tri- and tetrapyrophosphates; and cyclophosphates (also generally known in the art as “metaphosphates”). Useful monovalent cations of such phosphate salts include hydrogen, monovalent metals including alkali metals, and ammonium, for example. Synthetic anionic polycarboxylates may also be used in the dentifrice compositions of the present invention as an efficacy enhancing agent for certain active ingredients, including antibacterial, antitartar or other active agents within the oral composition. Such anionic polycarboxylates are generally employed in the form of their free acids or preferably partially or more preferably fully neutralized water-soluble alkali metal (e.g. potassium and preferably sodium) or ammonium salts. As discussed above, preferred copolymers are of maleic anhydride or acid with another polymerizable ethylenically unsaturated monomer, preferably methylvinylether/maleic anhydride having an approximate molecular weight (M.W.) of about 30,000 to about 2,500,000 most preferably about 30,000 to about 2,000,000. Examples of these copolymers are available from ISP corporation under the tradename Gantrez, e.g. AN 139 (M.W. 1,100,000), AN 119 (M.W. 200,000); S-97 Pharmaceutical Grade (M.W. 1,500,000), AN 169 (M.W. 2,000,000), and AN 179 (M.W. 2,400,000); wherein the preferred copolymer is S-97 Pharmaceutical Grade (M.W. 1,500,000).

The anionic polycarboxylate, is employed in certain embodiments in amounts effective to achieve the desired enhancement of the efficacy of any antibacterial, antitartar or other active agent within the dentifrice composition, Generally, the anionic polycarboxylates is present within the dentifrice composition from 0.05% to, 5% by weight, preferably from 0.5% to 2.5% by weight.

Orally acceptable carriers for use in the invention include the usual components of hydrogels, toothpastes, tooth powders, prophylaxis pastes, mouth rinses, lozenges, gums and the like. Selection of specific carrier components is dependent on the desired product form, including dentifrices, rinses, gels, and confectionaries.

As recognized by one of skill in the art, the oral compositions of the present invention optionally include other materials, such as for example, viscosity modifiers, diluents, surface active agents, such as surfactants, emulsifiers, and foam modulators, pH modifying agents, abrasives, humectants, emollients, and moisturizers, mouth feel agents, sweetening agents, flavor agents, colorants, preservatives and combinations thereof. It is understood that while general attributes of each of the above categories of materials may differ, there may be some common attributes and any given material may serve multiple purposes within two or more of such categories of materials.

In the present invention, it is particularly preferred that for the oral, preferably dental, application thereof of the antiseptic composition of the present invention is a gel developed by means of the optimal combination of the xanthone or xanthone compound according to the invention, preferably 9-hydroxycalabaxanthone or salts, solvates, stereoisomers or enantiomers thereof (HCX), and a hydrogel consisting of hyaluronic acid (HA) modified by means of chemical crosslinking or chemical gelling, allowing the controlled release of HCX, resulting in a gel with antibacterial and regenerative activity, that is biocompatible. Products currently found on the market have antibacterial activity but are toxic for tissue.

Native HA can be chemically functionalized to produce chemically reactive HA derivatives. Modifying HA consists of incorporating amino groups onto one batch of HA (HA-Amino) and creating aldehyde groups in another batch of HA (HA-Aldehyde) which react chemically when combined and are crosslinked to form the hydrogel which is relatively stable at physiological pH. One advantage of this system is that the precursor solutions of the gel can be applied as liquid to form the gel in situ. Furthermore, this configuration offers the possibility of formulating hydrogels with different mechanical properties by only varying the amounts or ratios of HA-Amino/HA-Aldehyde. With this approach, the results show that the HA hydrogel obtained is very stable, readily obtained in very mild conditions and at room temperature. The results show that despite the chemical modifications made on HA, its biocompatibility and regenerative properties are not lost, and it is not cytotoxic. In addition, the results of the formulation of the HA-HCX hydrogel show that the modified HA hydrogel absorbs large amounts of water (FIG. 6A) and that a controlled release of HCX into the aqueous medium occurs, maintaining a moderately sustained release of 20 μM after 10 h and up to 7 days (FIG. 6B). The quantification of released HCX shows that the concentration released and accumulated in one week is 120 μM. In addition, the results of FIG. 7 show that the gel modified with HCX has good antimicrobial properties, with a higher inhibition for a concentration of 0.05% and is similar to the effect produced by a commercial gel containing 0.2% chlorhexidine and unmodified hyaluronic acid (Periokin Hyaluronic 1%, Laboratorios Kin, Barcelona, Spain, HA 0.2% CHX). Therefore, in addition to the prolonged antibacterial activity, there are no cytotoxic effects on cells, and there is a regenerative effect due to the HA hydrogel.

It is noted that the present invention is not limited to gels of hyaluronic acid, although this is specially preferred, but any further hydrated gels are also suitable for the present invention. For example, further hydrogel gels useful in the present invention can be any type of hydrated gels that can be in turn classified according to their behavior towards water, into hydrophobic gels or lipogels and hydrophilic gels. Furthermore, according to the phases, they can be differentiated into single-phase gels or two-phase gels, or according to their viscosity into fluid, semi-solid or solid gels. According to the polymers used, gels useful in the present invention can be classified into:
Natural:
   Vegetable: i.e. gum arabic, gum karaya, gum tragacanth.
   Seed extracts: i.e. guar gum, carob “carubin” gum, starch and derivatives, pectins.
   Leaf and flower extracts: i.e. mallow mucilage, altea, calendula, plantain.
   Marine origin: i.e. Agar-agar gum, Carrageenans (Irish moss), Alginates.
   Animal origin: i.e. Casein, Gelatin
   Exocellular hydrocolloids: Xanthan Gum
Modified natural polymers:
   Cellulose ester derivatives: i.e. Methylcellulose, Ethylcellulose, Ethylmethylcellulose, Ethylhydroxyethylcellulose, Hydroxyethylcellulose, Hydroxyethylmethylcellulose, Hydroxypropylcellulose, Hydroxypropylmethylcellulose, Sodium carboxymethylcellulose, Sodium sulfate cellulose, Carboxymethylhydroxyethylcellulose, Alkyl cellulose, Quaternized hydroxyethylcellulose.
   Guar derivatives: i.e. hydroxy propyl guar (JAGUAR HP 8@), carboxy methyl hydroxy propyl guar, hydroxy propyl trimonium guar chlorhydrate
Vinyl polymers or copolymers: polyvinyl alcohols, polyvinyl pyrrolidone (PVP), PVP/PVA (polyvinyl acetate) and PVA/Ac. crotonic copolymers, polyvinyl methyl ether, methyl vinyl ether-malic anhydrous copolymer (MVE/MA), quaternary polyvinyls
Carboxyvinyl polymers: carbomer (Carbopol®)
Acrylic polymers—Acrylamides—: carboxypolyacrylic acid, methyl acrylate, ethyl acrylate, methyl methacrylate and methacrylic polymer, acrylic copolymer, acrylic interpolymer, polyacrylamide, dimethyl idantoinformaldehyde, imidazolinylurea
Fatty acid esters of modified steroisomeric structure: 12-oxystearic triglycerides, aluminum hydroxystearate.
Pyrogenic and precipitated silica anhydride—pyrogenic aluminum and titanium oxide: pyrogenic silica, precipitated silica, pyrogenic aluminum oxide, pyrogenic titanium oxide
Alkaline earth silicate of colloidal lamellar structure or derivatives with quaternary traces for polymerization: bentonite, montmorillonite, atalpulguite, quaternized organophilic bentonite.

Therefore, in another preferred embodiment, the antiseptic composition of the present invention is in the form of a hydrogel, wherein the xanthone or xanthone compound according to the invention, preferably 9-hydroxycalabaxanthone or salts, solvates, stereoisomers or enantiomers thereof (HCX), is entrapped in the hydrogel. Apart from HA, other particularly useful hydrogels are those shown in example 12 such as those produced by using gelatin Methacryloyl (GelMA), alginate or hydroxymethyl propyl cellulose.

Preferably, the antiseptic composition of the present invention is in the form of a hydrogel comprising water, a hyaluronic acid derivative and xanthone or xanthone compound according to the invention, preferably 9-hydroxycalabaxanthone or salts, solvates, stereoisomers or enantiomers thereof (HCX), wherein:
a. the hyaluronic acid derivative comprises hyaluronic acid, or salts, solvates or stereoisomers, preferably pharmaceutically acceptable salts, solvates or stereoisomers thereof, preferably of a molecular weight comprised between 50,000 and 3,500,000 Da;
b. the concentration of said hyaluronic acid derivative or salt thereof is preferably comprised between 0.001% and 5%; and
c. the xanthone or xanthone compound according to the invention, preferably 9-hydroxycalabaxanthone or salts, solvates, stereoisomers or enantiomers thereof (HCX), is entrapped in the hydrogel and has preferably a concentration comprised between 1 μM, 10 μM, 25 μM, or 50 μM and 5 mM or 3 mM, more preferably between 1 μM, 10 μM, 25 μM, or 50 μM and 3.5 mM, more preferably between 1 μM, 10 μM, 25 μM, or 50 μM and 3 mM, more preferably between 1 μM, 10 μM, 25 μM, or 50 μM and 2 mM, more preferably between 1 μM, 10 μM, 25 μM, or 50 μM and 1 mM, more preferably from between 1 mM to 5 mM, more preferably from between 1 mM to 3.5 mM, more preferably from between 1 mM to 3 mM, more preferably about 3 mM.

Preferably in the case of the antiseptic composition being in the form of hydrogel gels that are applied to and left on the skin, for the treatment of diseases such as acne, impetigo, psoriasis, erysipelas, necrotizing fasciitis, cellulitis, folliculitis, furuncles and carbuncles, scalded skin syndrome, etc. it is preferred that the xanthone or xanthone compound according to the invention be present in the antiseptic composition of the present invention in an amount of about 0.2% to about 0.0002% by weight/volume, preferably about 0.05% to about 0.005% by weight/volume of the total composition. On the other hand, in the case of the antiseptic composition being in the form of hydrogel gels to be used as antibacterial gels such as periodontal gels or aft as it is preferred that a xanthone or xanthone compound according to the invention be present in said compositions in an amount of about 0.2% w/v to about 0.0002% w/v, preferably about 0.05% to about 0.005% w/v of the total composition. In another preferred embodiment, the antiseptic composition of the present invention, when present in the form of a hydrogel containing or comprising hyaluronic acid, the hyaluronic acid is at temperatures inducing gelling (physical crosslinking) and/or covalently crosslinked by chemically functionalized groups reactive to polymerization, preferably by nucleophilic addition reaction.

In another preferred embodiment, the antiseptic composition of the present invention, when present in the form of a hydrogel containing or comprising hyaluronic acid, the hyaluronic acid or derivative thereof is chemically functionalized to become reactive to polymerization or crosslinking, preferably by nucleophilic addition reaction.

In another preferred embodiment, the antiseptic composition of the present invention, when present in the form of a hydrogel containing or comprising hyaluronic acid, the hyaluronic acid or derivative thereof is crosslinked by the mix of two lots of hyaluronic acid where the first of them is functionalized preferably with adipic acid dihydrazide and the second of them is functionalized with aldehyde groups by the oxidation of vicinal hydroxyl groups, preferably with sodium periodate.

In the case of other useful hydrogels, as those shown in example 12, can be prepared as illustrated in said example. For example, Gelatin Methacryloyl (GelMA) hydrogels can be prepared as shown in example 12 by UV irradiation of the corresponding GelMA (containing the xanthone compound of the present invention) in the presence of a crosslinker. Alginate or hydroxymethyl propyl cellulose hydrogels can be also prepared as illustrated in example 12.

In various embodiments, and as already indicated, any of the oral compositions of the present invention, including the hydrogels, can be used in a method of promoting oral health in an oral cavity and for treating periodontal diseases, plaque, caries, halitosis, gingivitis, mucositis, mycosis, and oral aphthous ulcers, on an oral surface of a mammalian subject. Preferably, the disease is periodontitis and the related microbes are Porphyromonas gingivalis, Aggregatibacter actinomycetemcomitans, Tannerella forsythia, Treponema denticola, Fusobacterium nucleatum, Streptococcus mutans preferably at a concentration of between 1 μM, 10 μM, 25 μM, or 50 μM and 0.5 mM, more preferably between 1 μM, 10 μM, 25 μM, or 50 μM and 0.2 mM, more preferably between 1 μM, 10 μM, 25 μM, or 50 μM and 0.15 mM, more preferably between 50 μM and about 0.1 mM. Again, in the case of the antiseptic composition being in the form of hydrogel gels to be used as antibacterial gels such as periodontal gels or aft as it is preferred that a xanthone or xanthone compound according to the invention be present in said composition in an amount of about 0.2% w/v to about 0.0002% w/v, preferably about 0.05% to about 0.005% w/v of the total composition. Moreover, in the case of an antibacterial oral hygiene composition in aqueous form (such as mouthwashes and solutions for halitosis), it is preferred that the xanthone or xanthone compound according to the invention be present in said compositions in an amount of about 0.0001% w/v to about 0.1% w/v of the total composition. The method comprises preparing an oral care composition comprising the antiseptic composition of the present invention. Then, the oral care composition is contacted with one or more oral surfaces of the oral cavity. In certain embodiments, when the oral composition is contacted with one or more oral surfaces in the oral cavity, the oral composition has the effect of reducing inflammation of oral tissue.

Thus, in various embodiments of the present invention, the oral care composition that is prepared is a hydrogel, gel, dentifrice, confectionary, or mouthwash, the oral composition is preferably applied regularly to an oral surface, preferably on a daily basis, at least one time daily for multiple days, but alternately every second or third day. Most preferably the oral composition is applied to the oral surfaces from 1 to 3 times daily, at a pH of 4.5 to 10, generally 5.5 to 8, preferably 6 to 8, for at least 2 weeks up to 8 weeks or more up to lifetime.

In other various embodiments, the antiseptic composition of the invention is for the application thereof on body surfaces such as the skin or mucosae, for example, in the treatment of medical conditions associated with infection such as acute or chronic wounds, burns, acne, impetigo, psoriasis, erysipelas, necrotizing fasciitis, cellulitis, folliculitis, furuncles and carbuncles, scalded skin syndrome, and surgical wounds, or disinfection of the skin before a surgical procedure, different HCX compositions are herein described for this purpose and their antimicrobial activity and biocompatibility tested in a healthy 3D skin tissue.

Finally, in other various embodiments, the antiseptic composition of the invention is for the application as a disinfectant formulation or solution for disinfecting a substrate surface. In this respect, the disinfectant formulation may be used for destroying microorganisms or a virus and/or inhibiting the growth of microorganisms or a virus on any substrate surface, such as a disinfecting solution for a soft contact lens. Commonly, the microorganisms are bacteria. The invention also relates to the disinfectant formulation or solution for use in a method of disinfecting and/or sterilizing any medical devices, contact lenses or any other device for use in the cosmetic or pharmaceutical industry, comprising treating the devices with the disinfectant solution of the invention.

As stated previously, the disinfectant formulation of the present invention may be suitable for disinfecting any substrate surface against a number of microorganisms including gram positive bacteria or gram-negative bacteria. The disinfectant formulation of the present invention may be applied to a substrate surface in any environment in which said surface is likely to come into contact with a microorganism or virus. Such environments include healthcare environments such as hospitals, clinics, and nursing homes, schools, sport facilities, hospitality, public transport, agriculture, manufacturing, residential homes, and cruise ships. Preferably, the disinfectant formulation is used in a healthcare environment such as a hospital. The substrate surface may be made of any material. In particular, substrate surfaces on which the disinfectant formulation of the present invention may be applied include wood, metal, laminates, plastics/polymer surfaces, ceramic etc.

The following Examples further illustrate the present invention, but it is understood that the invention is not limited thereto. All amounts and proportions referred to herein and in the appended claims are by weight unless otherwise indicated.

### EXAMPLES

### Example 1. Evaluation of the Antimicrobial Activity of HCX on P. Gingivalis and of the Cytotoxicity Thereof in Gingival Fibroblasts

### Material and Methods

### 1.1. Different Concentrations of HCX Used

Three different concentrations of HCX (0.00002, 0.0002, 0.002%) were used for studies with gingival fibroblasts and bacteria. Treated cells without the biomolecule served as negative control (C−) and chlorhexidine (CHX) (Abcam, Cambridge, United Kingdom) at 0.001% served as positive control.

### 1.2. Gingival Fibroblast Culture

Immortalized human gingival fibroblasts (iHGF) (Applied Biological Materials Inc, Richmond, BC, Canada) were cultured at 37° C. in a 5% CO₂ atmosphere using a fibroblast medium consisting of low glucose Dulbecco's Modified Eagle Medium (DMEM) (Biowest, Nuaille, France) and Ham's F-12 medium (3/1) (Biowest), supplemented with 10% (v/v) embryonic stem cell fetal bovine serum (FBS) (Biowest) and 100 μg/ml penicillin, and 100 μg/ml streptomycin (Biowest). The culture medium was refreshed twice a week. Cells were seeded in 48-well plates at a density of 2×10⁴ cells/well. When the cells reached confluence, they were treated with different concentrations of HCX, and cytotoxicity was determined.

1.3. Treatment with Different Concentrations of HCX

48 hours after seeding the iHGF cells in 48-well plates, cell inflammation was induced by means of adding 1 μg/ml of P. gingivalis lipopolysaccharide (LPS) (InvivoGen, San Diego, CA, USA) and the cells were treated with different concentrations of HCX (0.00002, 0.0002, 0.002%) in a fibroblast medium with 50 μg/ml of ascorbic acid (Sigma-Aldrich, St. Louis, MO, USA) for 48 hours. Images of the cells were taken with a bright field inverted microscope (Nikon Eclipse TS100) before treatment and 24 hours and 48 hours after treatment.

### 1.4. Cell Cytotoxicity

To estimate the cytotoxicity of the different concentrations of HCX, the presence of lactate dehydrogenase (LDH) in the culture media 48 h after pro-inflammatory stimulation with LPS and treatment with HCX was used as the cell death index. Following the manufacturer's instructions (Cytotoxicity Detection kit, Roche Diagnostics, Mannheim, Germany), LDH activity was determined spectrophotometrically after a 30-minute incubation at room temperature (RT) of 50 μl of culture media and 50 μl of the reaction mixture, measuring nicotinamide adenine dinucleotide (NADH) oxidation at 490 nm in the presence of pyruvate. Results were presented in relation to LDH activity of the media of the cells seeded without treatment (negative control, 0% cell death) and in relation to cells treated with 1% Triton X-100 (positive control, 100% death) using the following equation: Cytotoxicity (%)=[(expected value−negative control)/(positive control−negative control)]×100. The assays were performed in triplicate, with two replicates in each condition (n=6).

### 1.5. Metabolic Activity of the Cells

Total metabolic activity was evaluated after treating the cells for 48 h with different concentrations of HCX. Presto Blue reagent (Life Technologies, Carlsbad, CA) Was used, incubating the reagent for 1 h following the manufacturer's protocol. Untreated cells were set as 100% cell viability. The assays were performed in triplicate, with two replicates in each condition (n=6).

1.6. Bacterium P. gingivalis Culture and Proliferation Assay

Bacterium P. gingivalis (ATCC 33277TM, Manassas, VA) was cultured in brain heart infusion (BHI) broth (Scharlab, Barcelona, Spain), supplemented with 0.5 g/l of L-cysteine hydrochloride (Thermo Fisher Scientific), 5.0 mg/I of hemin (Thermo Fisher Scientific), and 1.0 mg/I of vitamin K (Thermo Fisher Scientific) in anaerobic conditions (10% H₂, 10% CO₂, and 80% N₂) achieved with an anaerobiosis sachet and jar (Oxoid Anaerogen™, Thermo Fisher Scientific) at 37° C. for 24-72 h.

Bacteria P. gingivalis were cultured from existing frozen bacteria in BHI broth. After one night of incubation, 1 ml of bacterial suspension (˜3×10⁸ bacteria/ml) was treated with different concentrations of HCX (0.00002, 0.0002, 0.002%) for 10 hours in anaerobic conditions (10% H₂, 10% CO₂, and 80% N₂) at 37° C. At regular intervals (0 h and 10 h), optical density (OD) was measured at 600 nm to determine bacterial proliferation (PowerWave Ht, Biotekinstruments, Winooski, VT). Bacterial growth rate (μ) was calculated for the exponential phase of growth following the equation ln ODₜ−ln OD₀=μ−(t−t₀); the assay was performed in triplicate, with two replicates in each condition (n=6). The proportion of living bacteria/dead bacteria was determined using the LIVE/DEAD BacLight bacterial viability kit (Invitrogen, Thermo Fisher Scientific, Waltham, MA, USA), following the manufacturer's instructions. The assays were performed in triplicate, with two replicates in each condition (n=6).

### Results

Individually, in experiments with human gingival fibroblast cells, cytotoxicity evaluated by means of the activity of lactate dehydrogenase (LDH) released into the medium is not observed (FIG. 2).

Results of the evaluation of the antimicrobial properties of HCX with respect to P. gingivalis show that a concentration of 0.002% is sufficient to eliminate 100% of the bacteria (FIG. 3).

### Example 2. Production of Modified HA and Incorporation of HCX

### Material and Methods

### 2.1 Synthesis of the HA-Amino (HA-ADH) Derivative

In a typical reaction, 500 mg (1.32 mmol; Mw=1.2×10⁶ g/mol) of HA were dissolved in milliQ H₂O in a concentration of 3 mg/ml at room temperature. A 30-fold molar excess of adipic acid dihydrazide (ADH) (6887.9 mg; 39.54 mmol) was then added, and when it was completely dissolved, the pH of the reaction mixture was adjusted to 6.8 with solutions consisting of [NaOH]=0.1 μM and [HCl]=0.1 M. A mixture formed by 1012.2 mg (5.28 mmol) of EDC/735.5 mg (5.28 mmol) of 1-hydroxybenzotriazole (HOBt) dissolved in 5 ml of DMSO/H₂O 1:1 was then added. The reaction was carried out overnight in darkness at room temperature and controlling the pH=6.8 by means of adding [NaOH]=0.1 M. After the reaction time, the pH was adjusted to 7.0 and the product was subjected to dialysis at 37° C. for 7 days. NaCl was then added to the product until having a 5% solution (w/v), and the product was precipitated using three equivalent volumes of ethanol. The precipitate was then recovered, redissolved in milliQ H₂O at a concentration of 5 mg/ml, and lyophilized for one week. Finally, the product was stored at −70° C. under N₂ atmosphere.

### 2.2 Synthesis of the HA-Aldehyde (HA-CHO) Derivative

In a typical reaction, 1000 mg (2.64 mmol; Mw=1.2×10⁶ g/mol) of HA were dissolved in milliQ H₂O at a concentration of 3 mg/ml at room temperature. Next, 21 ml of a solution in milliQ H₂O of 563.9 mg (2.64 mmol) of sodium periodate (NaIO₄) were added dropwise. The reaction was carried out for 2 h at room temperature in darkness. Next, 671 μl (12 mmol) of ethylene glycol were added to inactivate any unreacted periodate. The solution was subjected to dialysis for one week and lyophilized for one week. The product was stored at −70° C. under N₂ atmosphere.

2.3 Synthesis of HA Hydrogels with HCX

Two solutions of [HCX]=0.025 and 0.05% in milliQ H₂O were prepared. Then, HA-ADH and HA-CHO were dissolved separately in each of the aqueous solutions of [HCX] at a concentration of 7 mg/ml each. Hydrogels with HCX were formed by mixing the HA-ADH and HA-CHO solutions.

### Results

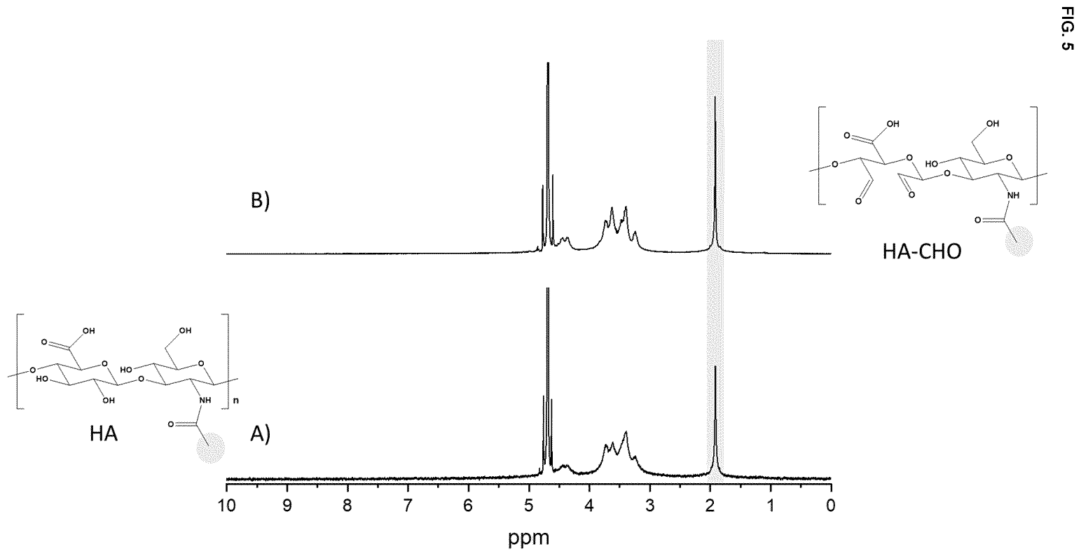

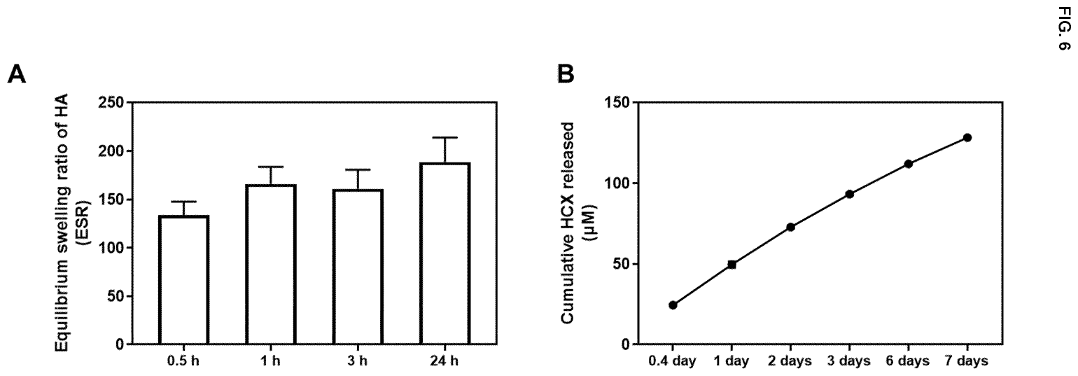

The bars of FIG. 4 show the signals characteristic of the functional groups present in HA before and after chemical modification with ADH (FIG. 3A and FIG. 3B). HA is a polymer with a very high molecular weight (Mw=1.2×10⁶ g/mol), hence the existence of the broad multiplet between about δ=3.0-3.9 ppm which corresponds to the signal of the protons of the sugar rings. The signal a δ=1.89 ppm highlighted by the green bar corresponds to the N-acetyl functional group of methyl and was used as the internal standard for calculating the degree of modification (DM) of the HA since this signal is rarely modified during the synthesis of HA derivatives. In this case, the DM was 45%, a good result taking into account the high molecular weight of the polymer of the present invention. Signals at δ=1.53 ppm, δ=2.14 ppm, and δ=2.27 ppm highlighted by red, orange, and purple bars, respectively, correspond to the methylene groups of ADH and confirmed the successful chemical modification of HA by ADH. Furthermore, through NMR experiments for HA and HA-CHO (FIG. 5), small changes were able to be confirmed in the region of the protons of the sugar rings, which allow confirming a new chemical structure in HA. Furthermore, the viscosity of the reaction mixture decreased visually during the reaction time, indicating the oxidation of HA and the slight loss of molecular weight typical in these processes.

Results of the formulation of the HA-HCX hydrogel show that the modified HA hydrogel absorbs large amounts of water (FIG. 6A) and that a controlled release of HCX into the aqueous medium occurs, maintaining a moderately sustained release of 20 μM after 10 h and up to 7 days (FIG. 6B). The quantification of released HCX shows that the concentration released and accumulated in one week is 120 μM.

### Example 3. Effect of the Modified HA Hydrogel with HCX on the Growth and Viability of P. gingivalis

### Material and Methods

3.1. Different Concentrations of HA Formulated with HCX.

Two different concentrations of HA formulated with HCX (0.025 and 0.05%) were used in the present study. Cells treated with HA without biomolecule served as negative control (HA) and cells treated with the commercial gel Periokin Hyaluronic 1% (Laboratorios Kin, Barcelona, Spain) containing 0.2% CHX and 1% HA (HA 0.2% CHX) served as positive control. Gels at a concentration of 5% (v/v) were used for bacterial studies.

3.2. Bacterium P. gingivalis Culture and Proliferation Assay with Different Concentrations of Modified HA with HCX.

The bacteria P. gingivalis were cultured and treated with different concentrations of HA formulated with HCX (0.025 and 0.05%), like in experiment 1 of section 1.6.

### Results

The results of FIG. 7 show that the gel modified with HCX has good antimicrobial properties, with a higher inhibition for a concentration of 0.05% and is similar to the effect produced by a commercial gel containing 0.2% chlorhexidine and unmodified hyaluronic acid (Periokin Hyaluronic 1%, Laboratorios Kin, Barcelona, Spain, HA 0.2% CHX).

### Example 4. Effect of the Modified HA Hydrogel with HCX on the Viability of a 3D Gingival Tissue

### Material and Methods

### 4.1. Cell Culture

Immortalized human gingival keratinocytes (iHGK) (Applied Biological Materials Inc) were cultured in culture flasks for sensitive adherent cells (Sarstedt, Germany) at 37° C. in a 5% CO₂ atmosphere using a keratinocyte medium consisting of calcium and magnesium-free DMEM (Gibco, Grand Island, NY, US)/Ham's F12 (3/1) (Biowest), supplemented with 0.01 mg/ml of insulin (Sigma-Aldrich), 0.4 ng/ml of hydrocortisone (Sigma-Aldrich), 6.7 ng/ml of selenium (Sigma-Aldrich), 0.01 μg/ml of human epithelial growth factor (Thermo Fisher Scientific, Waltham MA, USA), 1 M HEPES buffer (Biowest), 5.5 μg/ml of transferrin (Sigma-Aldrich), 10⁻¹⁰ M of cholera toxin (Sigma-Aldrich), 2 mM of L-glutamine (Sigma-Aldrich), 5% (v/v) of FBS (Biowest), and 100 μg/ml of penicillin, and 100 μg/ml of streptomycin (Biowest). The culture medium was refreshed twice a week.

iHGFs were cultured as described in Experiment 1, section 1.2. Both iHGK cultures and iHGF cultures with 70-80% confluence were used for constructing 3D tissue, as described in section 4.2 below.

### 4.2. 3D Gingival Tissue Construction

3D gingival tissue was constructed according to the technique described by Dongari Bagtzoglou and Kashleva. In summary, a rat tail collagen type I solution (Thermo Fisher Scientific) (2.2 mg/ml) was mixed with iHGFs (1×10⁵ cells/well) and pipetted into a 24-well insert (Sarstedt) with pores measuring 0.4 mm. The collagen with fibroblasts was cultured at 37° C., 5% CO₂, for 7 days in a fibroblast medium. Next, iHGKs were seeded (2.5×10⁵ cells/well) on top of 3D tissues and cultured in a keratinocyte medium for 3 days at 37° C., 5% CO₂. Next, the 3D tissues were cultured in the air-liquid interphase at 37° C., 5% CO₂, for 15-17 days in an airlift (AL) culture medium consisting of low glucose DMEM (Biowest)/Ham's F12 (3/1) (Biowest), supplemented with 5 μg/ml of insulin (Sigma-Aldrich), 0.4 μg/ml of hydrocortisone (Sigma-Aldrich), 2×10⁻¹¹ μM of 3,3′,5-triiodo-L-thyronine (T3) (Sigma-Aldrich), 1.8×10⁻⁴ M of adenine (Sigma-Aldrich), 5 μg/ml of transferrin (Sigma-Aldrich), 10⁻¹⁰ M of cholera toxin (Sigma-Aldrich, St. Louis, MO, USA), 2 mM of L-glutamine (Sigma-Aldrich), 5% (v/v) of FBS (Biowest), and 100 μg/ml of penicillin, and 100 μg/ml of streptomycin (Biowest). The AL medium was changed every two days. After 25 days, the tissues were treated with modified HA gels with HCX.

4.3. Treatment of 3D Gingival Tissues with Modified HA Gels with HCX.

At the end of the 3D tissue incubation period, the tissues were surface treated with 50 μl of 1 μg/ml of P. gingivalis LPS (InvivoGen) for 24 h (FIG. 1B) to cause inflammation therein. Next, 30 μl of HA gel, 30 μl of HA with 0.025% of HCX, 30 μl of HA gel with 0.05% 30 μl of PBS (Biowest) (negative control), and 30 μl of the commercial gel Periokin (HA 0.2% CHX) (positive control) were applied on the tissue for 48 h. The assays were performed in triplicate, with two replicates in each condition (n=6).

### 4.4. MTT Assay

At the end of the incubation period with different HA gels, the tissues were washed with PBS (Biowest) and incubated with 400 μl of 0.5 mg/ml of MTT (3-(4,5-dimethylthiazol-2-yl)-2,5-diphenyltetrazolium bromide (Thermo Fisher Scientific). After 3 hours of incubation at 37° C. and 5% CO₂, the cultures were incubated with 2 ml of isopropanol (Sigma-Aldrich) for 24 h at RT. Optical density was measured with 200 μl of the extracts at 570 nm (reference filter: 690 nm). The negative control was obtained from the tissue culture media treated with PBS (100% viability). The positive control was obtained from the tissue culture media treated with 10% sodium dodecyl sulfate (SDS) diluted in PBS (1:1). Results are expressed as percentage of viability compared to the negative control. The assays were performed in triplicate, with two replicates in each condition (n=6).

### Results

The results of FIG. 8 show that the modified gel with HCX presents very good biocompatibility in a 3D gingival tissue, similar to modified HA without HCX and to an untreated gingival control tissue, for the two concentrations of 0.025 and 0.05%. However, the commercial gel containing 0.2% chlorhexidine and unmodified hyaluronic acid (Periokin Hyaluronic 1%, Laboratorios Kin, Barcelona, Spain, HA 0.2% CHX) presents toxic effects on the tissue, similar to the 5% SDS group used as positive control of 100% cell death.

### Example 5: Evaluation of the Antimicrobial Activity of HCX on Staphylococcus epidermidis

### Material and Methods

5.1. Culture and Proliferation Assay of S. epidermidis

S. epidermidis is a Gram-positive bacterium normally found on human skin and mucosa membranes. This bacterium can cause wound infections, boils, sinus infections, endocarditis, and other infections. S. epidermidis can survive for a long time on various surfaces—especially on polymeric plastic or metal—and form a biofilm that adheres well.

S. epidermidis 4814 (CECT, Valencia, Spain) were grown from frozen stocks in Luria-Bertani (LB) broth (Scharlab, Sentmenat), at 37° C. under aerobic conditions in an orbital shaker (180 rpm). After an overnight incubation, 1 mL of bacterial suspensions were incubated with different concentrations of HCX (0.003, 0.002, 0.001, 0.0002, 0.00002%) for 24 hours at 37° C. under aerobic conditions in an orbital shaker (180 rpm). Bacterial suspension without treatment served as negative control, and CHX at 0.001% served as positive control for bacterial growth inhibition. The optical density (OD) was measured at 600 nm at 0 h, 10 h and 24 h to determine bacterial proliferation (PowerWave Ht, Biotek instruments, Winooski, VT, USA). Bacterial growth rate (μ) was calculated during the exponential growth phase following the equation ln ODt−ln OD0=μ×(t−t0). Live/Dead ratio of bacteria was determined using the LIVE/DEAD BacLight bacterial viability kit (Invitrogen, Thermo Fisher Scientific, Waltham, MA, USA), following manufacturer's instructions. The assays were carried out in triplicate, with two replicates at each condition (n=6).

### Results

Some of the most common infectious diseases are caused by bacteria that naturally colonize human skin asymptomatically. Staphylococcus epidermidis is carried asymptomatically on the skin and mucous membranes of virtually all humans but is a major cause of nosocomial infection associated with invasive procedures.

In this example, we show that a concentration of 0.001% of HCX incubated for 10 h on S. epidermidis is sufficient to eliminate 100% of bacteria and a concentration of 0.003% of HCX incubated for 24 h on S. epidermidis eliminates 80% of bacteria, which is similar to the effectivity of the most common antiseptic used for the skin, chlorhexidine.

### Example 6: Effect of Hyaluronic Acid Gel Containing 0.05% HCX on the Bacteria Present in the Gingival Crevicular Fluid after Inducing Periodontitis in Rats with Lipopolysaccharide (LPS) from Porphyromonas gingivalis

### Material and Methods

### 6.1. In Vivo Experimental Protocol

In this study, Wistar rats aged 12 weeks and weighed 292-360 g were used. Experimental periodontitis was induced in all rats. A mixture of ketamine (anesketin)/xylazine (xylasol) 80/10 mg/kg was administered intraperitoneally to induce anaesthesia. Anaesthesia was maintained with 2-3% isoflurane in 100% oxygen using a small nose cone. The animal was placed on its back and the maxilla and mandible was stabilized in an open position with aluminium mouth gags. The ligature model was created by placing a sterile braided silk ligature (5/0) (Laboratorio Aragó, Barcelona, Spain) around the first maxillary molar bilaterally within the gingival sulcus using forceps and securing with the surgeon's knots on the lingual surface. Also, periodontitis was induced by injection of 40 μL of LPS (1 mg/mL) (InvivoGen, San Diego, CA, USA) derived from P. gingivalis in sterile saline, was injected 3 times per week with a microsyringe to the subgingival tissue at the mesio-palatal side of the first and second maxillary molars. The induction of periodontitis was maintained for 14 days to promote the accumulation of bacteria biofilm and consequent inflammation. Once the intervention was over, its effect was reversed with the antagonist atipamezole (antisedan 0.5 mg/kg) administered subcutaneously.

After 14 days of inducing periodontitis in the rats, animals were randomly divided into three treatment groups. Control was the group with no treatment (treatment A), HA with 0.05% of HCX gel applied group (treatment B) and HA with 0.2% of CHX commercial gel applied group (treatment C, PERIOKIN HYALURONIC 1% Gel). To be able to treat the animals with periodontitis with the different gels, the rats were under general anaesthesia. A mixture of ketamine (anesketin)/xylazine (xylasol) 80/10 mg/kg was administered intraperitoneally to induce anaesthesia. Anaesthesia was maintained with 2-3% isoflurane in 100% oxygen using a small nose cone. The animal was placed on its back and the maxilla and mandible must be stabilized in an open position with aluminium mouth gags. First, the ligatures were removed, and the surgical area was cleaned with saline. Next, an intracrevicular incision was made around a maxillary first molar with a microsurgical blade. The incision was extended to the mucosa of the anterior maxilla from the mesial surface of the molars to the posterior border of the incisive papilla. A full-thickness mucoperiosteal flap was raised. After hemostasis was achieved, 30 μl of the gel was applied to the wound. The flaps were repositioned, and the wound was sutured with a sterile braided silk ligature (6/0) (Laboratorio Aragó). Once the intervention was over, its effect was reversed with the antagonist atipamezole (antisedan 0.5 mg/kg) administered subcutaneously. The gel treatments were carried out topically for the next 14 days, 3 times a week, 30 μl of the gels were homogeneously distributed with sterile swabs in the lingual and vestibular region around the molars, under isoflurane anaesthesia with 2-3% induction and 1% maintenance. Gingival Crevicular fluid (GCF) was collected before inducing periodontitis (basal control), before treatment at the onset of periodontitis, and after 14 days of treatment. GCF was collected using adsorbent paper point no 30 (Proclinics. S.A.U, Barcelona, Spain) around mesio-palatal of the first molar. After collection, the paper point was immediately transferred into a plastic vial and stored at −80° C. until the assays. After 28 days of the study, the animals were sacrificed with CO2 (>70%) in carbon dioxide chamber. Following euthanasia, samples of GCF were taken with adsorbent paper point (Proclinics. S.A.U) to perform cytokine analyses and a metagenomic characterization and comparative microbiota analysis of the different groups, then the jaws were extracted and fixed in formalin for radiological and histological analysis.

### 6.2. GCF DNA Extraction, Library Preparation and Sequencing.

The 16S rRNA gene analysis was performed at Microomics (Barcelona, Spain; http://www.microomics.eu/). DNA was extracted from adsorbent paper points of GCF at different times, before inducing periodontitis (basal control) and after inducting periodontitis for 14 days (comparison 1). DNA was also collected after 14 days of the application of different treatments (comparison 2) using MagMAX CORE Nucleic Acid Purification Kit 500 RXN (Thermo Fisher, CA, Austin), following the manufacturer's instructions. As a control for downstream procedures, Mock community DNA was included (Zymobiomics Microbial Community DNA, ZymoResearch, Irvine). Samples were amplified using 16S rRNA V3-V4 regions specific primers (V3-V4-Forward 50-TCGTCGGCAGCGTCAGATGTGTATAAGAGACAGCCTACGGGNGGC WGCAG-30, V3-V4-Reverse 50 GTCTCGTGGGCTCGGAGATGTGTAT AAGAGACAGGACTACHVGGGTATCTAATCC-30).

The PCR was performed in 10 μL final volume with 0.2 μM primer concentration. The PCR cycle included: 3 minutes at 95° C. (initial denaturation) followed by 25 cycles: 30 seconds at 95° C. 30 s at 55° C., and 30 s at 72° C., and a final elongation step of 5 minutes at 72° C. PCR products were purified using AMPure XP beads (Beckman Coulter, Nyon, Switzerland) with a 0.9× ratio according to manufacturer's instructions. The above-described primers contain overhangs allowing the addition of full-length Nextera barcoded adapters for multiplex sequencing in a second PCR step, resulting in sequencing ready libraries with approximately 450 bp insert sizes. In brief, 5 μL of the first PCR purified product were used as template for a second PCR with Nextera XT v2 adaptor primers in a final volume of 30 μL using the same PCR mix and thermal profile as for the first PCR but with only 8 cycles. Twenty-five microliters of the second PCR product were purified with SequalPrep normalization kit (Invitrogen, ThermoFisher Scientific, Waltham, Massachusetts), according to the manufacturer's protocol. Libraries were eluted in 20 μL final volume and pooled for sequencing. The final pool was quantified by qPCR using Kapa library quantification kit for Illumina Platforms (Kapa Biosystems, SigmaAldrich, Saint Louis, Missouri) on an ABI 7900HT real-time cycler (Applied Biosystems, ThermoFisher Scientific, Waltham, Massachusetts). Sequencing was performed using Illumina MiSeq (2×300 bp) and v3 chemistry with a loading concentration of 10 PM In all cases, 15% of PhIX control libraries for run quality monitoring were used. Negative controls of the sample collection buffer, DNA extraction, and PCR amplification steps were routinely performed in parallel, using the same conditions and reagents, PCR products after both PCR steps were visualized by electrophoresis gel (1.5% agarose) with SYBR Safe (ThermoFisher Scientific, Waltham, Massachusetts).

### 6.3. Bioinformatics Processing and Statistical Analysis.

Raw demultiplexed forward and reverse reads were processed using the following methods and pipelines as implemented in QIIME2 version 2019.4 with default parameters unless stated. DADA2 was used for quality filtering, denoizing, pair-end merging and amplicon sequence variant calling (ASV) using qiime dada2 denoise-paired method. Q20 was used as quality threshold to define read sizes for trimming before merging (parameters: -p-trunc-len-f and -p-trunclen-r). Reads were truncated at the position when the 75th percentile Phred score felt below Q20: 287 bp for forward reads, and 224 bp for reverse reads. After quality filtering steps, average sample size was 27240 (comparison 1) and 14200 (comparison 2) reads and 999 (comparison 1) and 2591 (comparison 2) phylotypes were detected. ASVs were aligned using the qiime alignment mafft method. The alignment was used to create a tree and to calculate phylogenetic relations between ASVs using the qiime phylogeny fasttree method. ASV tables were subsampled without replacement in order to even sample sizes for diversity analysis using qiime diversity core-metrics-phylogenetic pipeline. ASV tables were used to calculate alpha diversity metrics: community richness (ASVs), Evenness (or Pielou's Evenness; a measure of community evenness) and Shannon's diversity index (quantitative measure of community richness). Alpha diversity comparisons were performed using Kruskal Wallis non-parametric test. ASV tables and phylogenetic data were used to calculate beta diversity metrics: Bray-Curtis (non-phylogenetic quantitative measure) and Jaccard (non-phylogenetic qualitative measure) dissimilarity matrices, as well as Unweighted Unifrac (phylogenetic qualitative measure) and Weighted Unifrac (phylogenetic quantitative measure) distance matrices. Principal coordinated analysis (PCoA) was the method used for exploring and visualizing similarity of data using the matrices described above. The significance of groups present in community structure was tested using analysis of similarities (ANOSIM) and Permanova tests. Permdisp test was used to identify location vs dispersion effects. Distance-based redundancy analysis (dbRDA) was used to explore which variables constrained PCoA ordinations. Model selection was done stepwise using forward direction and a permutation test using vegan package version 2.5-5. Taxonomic assignment of ASVs was performed using Bayesian Classifier trained with Silva database (i.e., 99% OTUs database). Differential abundance of taxa was tested using two methods: ANCOM and Kruskal Wallis non-parametric test on relative abundance of taxa (total sum of scale). After Kruskal Wallis test, Conover's test was added for pairwise comparison. Benjamini-Hochberg correction was used to control false discovery rate (FDR). The relative abundance of each taxon was calculated as the number of 16S rRNA sequences of a given taxon divided by the total number of 16S rRNA sequence in a participant's sample. Significant threshold was set at 0.05. BiodiversityR version 2.11-1, PMCMR version 4.3, RVAideMemoire version 0.9-7 and vegan version 2.5-5 packages were used for the different statistical analysis carried out.

### Results

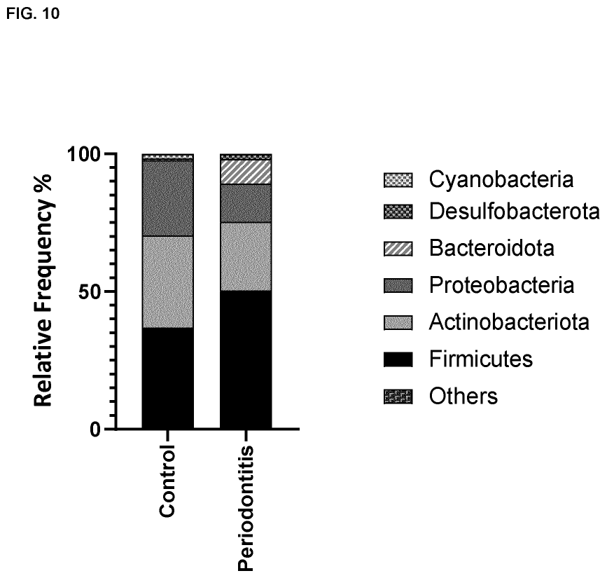

FIG. 10 shows the most abundant Phyla in healthy periodontium and in periodontitis disease in rats. The most abundance phyla in both groups of animals were Firmicutes, Actinobacteria, Proteobacteria and Bacteroidetes. Rats with periodontal disease presented an increase in the percentage of relative frequency of Bacteroidetes and Desulfobacterota accompanied by reduction in actinobacteria and proteobacteria relative abundance. Thus, the results confirmed the complexity of the oral microbiome, and the alterations occurred at the phylum level in the oral microbiome of rats with periodontal disease. When this microbiome suffers certain imbalances, a dysbiosis state generates a favorable environment for pathogenic microorganisms, increasing the virulence of microorganisms present.

Periodontitis-associated dysbiosis is characterized by low abundance or absence of specific bacteria normally present in healthy subgingival microbiota, and also by the appearance of pathogenic bacteria.

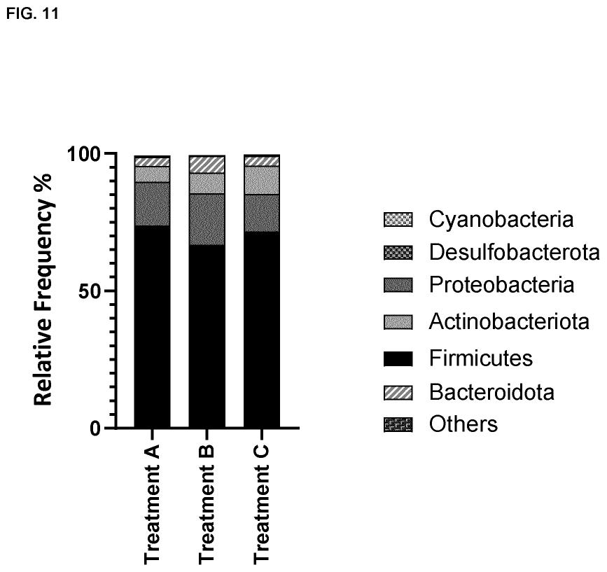

The oral microbiome also displayed differences in the most abundant phyla in periodontitis rats after applying the different treatments (A, B or C) (FIG. 11). The most abundance phyla in all groups of animals were Firmicutes, Actinobacteria, Proteobacteria and Bacteroidetes.

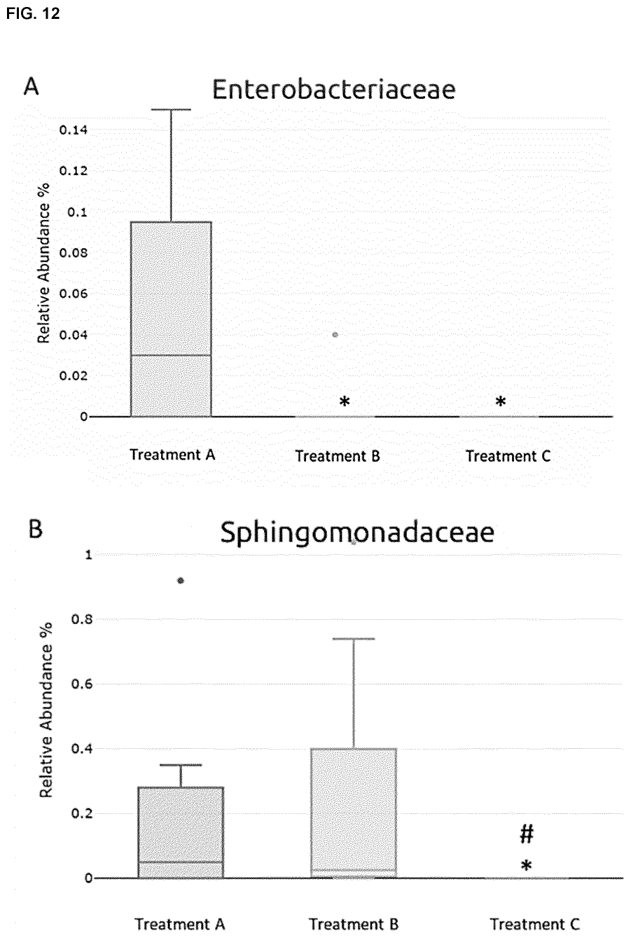

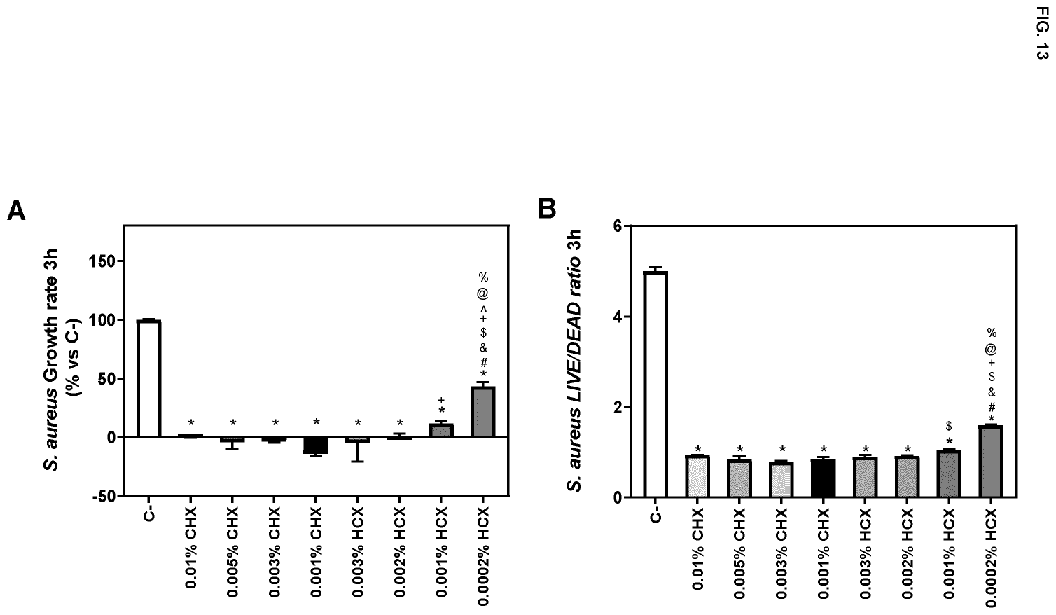

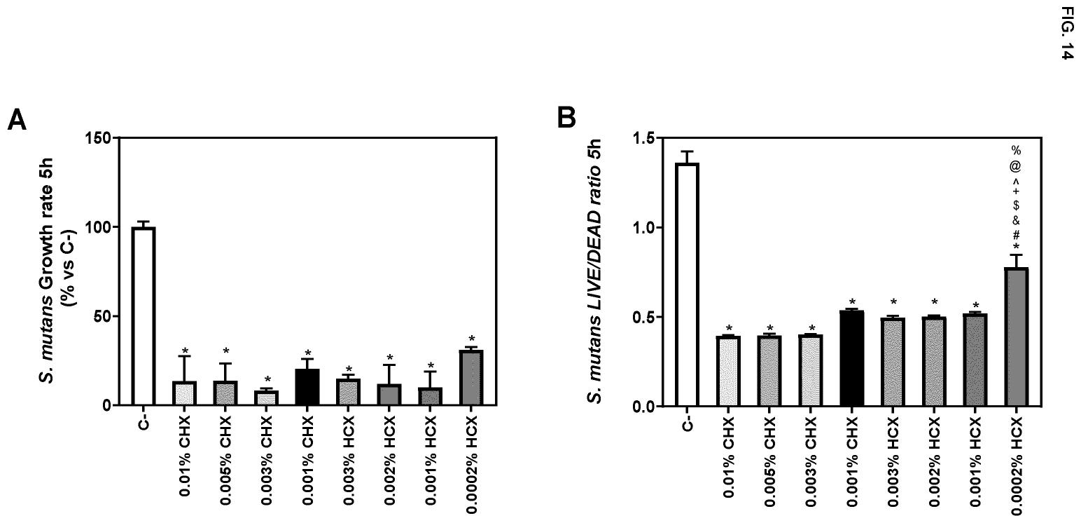

When comparing the different gel treatments, results demonstrate that Enterobacteriaceae (FIG. 12A) was significantly increased in samples from Treatment A (Control) compared to treatment B (animals with HA+0.05% HCX gel treatment) and C (animals with HA+0.2% CHX gel treatment). Microbiological studies in patients with periodontitis in different parts from the world have shown high prevalences of infection by Enterobacteriaceae, which could complicate the clinical picture of periodontal patients. On the other hand, Sphingomonadaceae, which is a specific bacterium present in the healthy subgingival microbiota (FIG. 12B), was significant decreased in samples from Treatment C compared to treatment A and B.

Our results show the HA gel containing HCX can help to re-stabilize the oral microbiome, by increasing the presence of bacteria related to a healthy oral microbiota, and by reducing the present of microbiota associated with periodontitis.

### Example 7: Evaluation of the Antimicrobial Activity of HCX on Staphylococcus aureus

### Material and Methods

7.1. Culture and Proliferation Assay of S. aureus

Staphylococcus aureus is a Gram-positive bacterium characterized by being the main cause of nosocomial bacteremia in different places of the world, due to the high virulence and pathogenicity factors.

S. aureus ATCC 29213 was grown from frozen stocks in Luria-Bertani (LB) broth (Scharlab), at 37° C. under aerobic conditions in an orbital shaker (180 rpm). After overnight incubation, 1 mL of bacterial suspensions were incubated with different concentrations of CHX (0.01, 0.005, 0.003, 0.001, 0.0001%) and HCX (0.003, 0.002, 0.001, 0.0002, 0.00002%) for 3 hours at 37° C. under aerobic conditions in an orbital shaker (180 rpm). Bacterial suspension without treatment served as negative control (C−). The optical density (OD) was measured at 600 nm at 0 h and 3 h to determine bacterial proliferation (PowerWave Ht). The bacterial growth rate (μ) was calculated during the exponential growth phase following the equation ln ODt−ln OD0=μ×(t−t0) (n=4). The live/Dead ratio of bacteria was determined using the LIVE/DEAD BacLight bacterial viability kit (Invitrogen), following the manufacturer's instructions. The assays were carried out in triplicate.

### Results

Staphylococcus aureus is a clinically important pathogen causing a wide range of human infections, from minor skin infections to severe tissue infections and sepsis. Moreover, S. aureus has a high level of resistance to antibiotics and is a common cause of infections in hospitals and the community, being a major health problem. This bacterium uses a wide range of virulence factors to develop an infection in the host.

In this example, we show that a concentration of 0.002% of HCX incubated for 3 h on S. aureus is sufficient to eliminate 100% of bacteria and a concentration of 0.001% of HCX incubated for 3 h on S. aureus eliminates 88% of bacteria, which is similar to the effectivity of the most common antiseptic used for the skin, such as chlorhexidine (CHX).

### Example 8: Evaluation of the Antimicrobial Activity of HCX on Streptococcus mutans

### Material and Methods

8.1. Culture and Proliferation Assay of S. mutans

Streptococcus mutans is a Gram-positive, facultative anaerobic bacterium that is normally found in the oral microbiota, which causes dental caries, bacteremia, and endocarditis.

S. mutans CECT 479 (obtained from The Spanish Type Culture Collection (CECT), University of Valencia) was grown from frozen stocks in BHI broth (Scharlab), at 37° C. under aerobic conditions in an orbital shaker (180 rpm). After overnight incubation, 1 mL of bacterial suspensions were incubated with different concentrations of CHX (0.01, 0.005, 0.003, 0.001, 0.0001%) and HCX (0.003, 0.002, 0.001, 0.0002, 0.00002%) for 5 hours at 37° C. under aerobic conditions in an orbital shaker (180 rpm). Bacterial suspension without treatment served as negative control (C−). The optical density (OD) was measured at 600 nm at 0 h and 5 h to determine bacterial proliferation (PowerWave Ht). The bacterial growth rate (μ) was calculated during the exponential growth phase following the equation ln ODt−ln OD0=μ×(t−t0) (n=3). The live/Dead ratio of bacteria was determined using the LIVE/DEAD BacLight bacterial viability kit (Invitrogen), following the manufacturer's instructions. The assays were carried out in triplicate, with two replicates at each condition (n=3).

### Results

S. mutans is a microorganism of the human oral cavity, forming part of dental plaque or dental biofilm. It is associated with the onset and development of dental caries and is the one that has the most influence on the development of the disease.

In this example, we show that a concentration of 0.001% of HCX incubated for 5 h on S. mutans is sufficient to eliminate 90% of bacteria and a concentration of 0.0002% of HCX incubated for 5 h on S. mutans eliminates 70% of bacteria, which is similar to the effectivity of the most common antiseptic used for the oral cavity, chlorhexidine.

### Example 9: Evaluation of the Antimicrobial Activity of HCX on Pseudomonas aeruginosa

### Material and Methods

9.1. Culture and Proliferation Assay of P. aeruginosa

Pseudomonas aeruginosa is a common, Gram-negative environmental organism. It has been associated with severe infections in humans, especially in patients with cystic fibrosis. These pathogens can cause serious therapeutic problems due to their natural resistance to antibiotics and ability to form biofilms.

P. aeruginosa ATCC 27853 was grown from frozen stocks in LB broth (Scharlab), at 37° C. under aerobic conditions in an orbital shaker (180 rpm). After overnight incubation, 1 mL of bacterial suspensions were incubated with different concentrations of CHX (0.01, 0.005, 0.003, 0.001, 0.0001%) and HCX (0.003, 0.002, 0.001, 0.0002, 0.00002%) for 3 hours at 37° C. under aerobic conditions in an orbital shaker (180 rpm). Bacterial suspension without treatment served as negative control (C−). The optical density (OD) was measured at 600 nm at 0 h and 3 h to determine bacterial proliferation (PowerWave Ht). The bacterial growth rate (μ) was calculated during the exponential growth phase following the equation ln ODt−ln OD0=μ×(t−t0) (n=3).

### Results

P. aeruginosa is one of the most important bacterial pathogens that causes infection with a high mortality rate due to resistance to different antibiotics. This bacterium prompts extensive tissue damage with varying factors of virulence, and its biofilm production causes chronic and antibiotic-resistant infections.

In this example, we show that a concentration of 0.003% of HCX incubated for 3 h on P. aeruginosa eliminates 32% of bacteria.

### Example 10: Evaluation of the Antifungal Activity of HCX on Candida albicans

### Material and Methods

10.1. Culture and Proliferation Assay of Candida albicans

C. albicans was grown from frozen stocks in LB broth (Scharlab), at 37° C. under aerobic conditions in an orbital shaker (180 rpm). After overnight incubation, the fungal suspension was sonicated for 5 s at 25 kHz then 1 mL of fungal suspensions were incubated with different concentrations of CHX (0.01, 0.005, 0.003, 0.001, 0.0001%) and HCX (0.003, 0.002, 0.001, 0.0002, 0.00002%) for 3 hours at 37° C. under aerobic conditions in an orbital shaker (180 rpm). Fungal suspension without treatment served as negative control (C−). The optical density (OD) was measured at 600 nm at 0 h and 25 h to determine fungal proliferation (PowerWave Ht). The fungal growth rate (μ) was calculated during the exponential growth phase following the equation ln ODt−ln OD0=μ×(t−t0) (n=3).

### Results

C. albicans is among the most prevalent fungal species of the human microbiota and asymptomatically colonizes healthy individuals. However, it is also an opportunistic pathogen that can cause severe, and often fatal, bloodstream infections. The medical impact of C. albicans typically depends on its ability to form biofilms, which attach to surfaces, such as tissues and implanted medical devices.

In this study, we show that a concentration of 0.003% of HCX incubated for 24 h on C. albicans eliminates 22% of fungal.

### Example 11: Evaluation of the Antimicrobial Activity of HCX on Streptococcus pyogenes

### Material and Methods

11.1. Culture and Proliferation Assay of S. pyogenes

Streptococcus pyogenes is a Gram-positive pathogen responsible for a wide range of infections, from uncomplicated diseases to more severe and invasive diseases with high morbidity/mortality and significant public health impact.

S. pyogenes was grown from frozen stocks in Tryptic Soy Broth (TSB) (Scharlab), at 37° C. under aerobic conditions in an orbital shaker (180 rpm). After overnight incubation, 1 mL of bacterial suspensions were incubated with different concentrations of CHX (0.01, 0.005, 0.003, 0.001, 0.0001%) and HCX (0.003, 0.002, 0.001, 0.0002, 0.00002%) for 24 hours at 37° C. under aerobic conditions in an orbital shaker (180 rpm). Bacterial suspension without treatment served as negative control (C−). The optical density (OD) was measured at 600 nm at 0 h and 24 h to determine bacterial proliferation (PowerWave Ht). The bacterial growth rate (μ) was calculated during the exponential growth phase following the equation ln ODt−ln OD0=μ×(t−t0) (n=4). The live/Dead ratio of bacteria was determined using the LIVE/DEAD BacLight bacterial viability kit (Invitrogen), following the manufacturer's instructions. The assays were carried out in triplicate.

### Results

Streptococcus pyogenes is one of the most important human bacterial skin pathogens, which can cause superficial impetigo or deeper cellulitis, but also more serious invasive infections such as sepsis, necrotizing fasciitis, and streptococcal toxic shock syndrome.

In this example, we show that a concentration of 0.0002% of HCX incubated for 24 h on S. pyogenes is sufficient to eliminate 100% of bacteria, which is similar to the effectivity of the most common antiseptic used for the skin, such as chlorhexidine (CHX).

### Example 12: Evaluation of Different Hydrogel Formulations with 0.05% HCX on Gingival Fibroblasts and on Staphylococcus epidermidis

### Material and Methods

12.1. Synthesis of Gelatin Methacryloyl (GelMA) and GelMA Hydrogel with 3 mM HCX

Gelatin Methacryloyl (GelMA) synthesis: In a typical experiment 1 g of gelatin (0.286 mmol lysine-functionalities) was dissolved in 60 mL PBS (1.66% w/w) at 50° C. separately, after 1 mL of methacrylic anhydride was added. The reaction was carried out at 50° C. for 3 h. Then, GelMA was dialyzed for 5 days at 37° C. and was lyophilized for 7 days. The product was stored at −70° C. under an N2 atmosphere.

GelMA hydrogel synthesis: The GelMA hydrogels were synthesized by UV irradiation of the corresponding GelMA in milliQ H2O with and without 0.05% of HCX (10% w/w) in the presence of irgacure 12959 (5% w/w respect to GelMA) as a crosslinker for 1.5 min. The UV lamp used for the hydrogel synthesis was a UVM-57 Handhel UV Lamp of 6 Watt and wavelength of 302 nm (Upland, CA, USA). The syntheses were carried out under irradiation at a constant distance of 2 cm from the lamp.

12.2. Synthesis of Alginate Hydrogel with 0.05% HCX

Alginate (FMC Biopolymers, Protanal LF200M, Norway) hydrogel was prepared overnight at 25° C. in milliQ H2O with and without 3 mM of HCX at 2% (w/v).

12.3. Synthesis of Hydroxymethyl Propyl Cellulose Hydrogel with 0.05% HCX

Hydroxymethyl propyl cellulose (HPMC) (Acofarma, Spain) hydrogel was prepared overnight at 25° C. in slow stirring in milliQ H2O with and without 0.05% of HCX at 2% (w/v) with Propylene glycol at 20% (w/v).

12.4. Synthesis of Modified Hyaluronic Acid (HA) Hydrogel with 0.05% HCX

Modified HA hydrogel with and without 0.05% HCX was synthesized like in experiment 2, section 2.3.

12.5. Evaluation of the Antimicrobial Activity of Different Hydrogels Formulations with 0.05% HCX on S. Epidermidis and of the Cytotoxicity Thereof in Gingival Fibroblasts

Different hydrogel formulations with and without 0.05% of HCX (HA, GelMa, Alginate and HPMC) and commercial gel containing 0.2% chlorhexidine and unmodified HA (Periokin Hyaluronic 1%, Laboratorios Kin, Commercial HA 0.2% CHX) were used for studies with gingival fibroblasts and S. epidermidis. Cells and bacteria without hydrogels served as negative control (C−). Gels at a concentration of 5% (v/v) were used for cell and bacterial studies.

Immortalized human gingival fibroblasts were cultured like in experiment 1 of section 1.2. The cells were treated with different hydrogel formulations with and without 0.05% of HCX (HA, GelMa, Alginate and HPMC) and commercial gel containing 0.2% chlorhexidine and unmodified HA (Periokin Hyaluronic 1%, Laboratorios Kin, commercial HA 0.2% CHX) in a fibroblast medium for 24 hours (n=4). Cell cytotoxicity was measured, like in experiment 1 of section 1.4.

The bacteria S. epidermidis was cultured and treated with different hydrogel formulations with and without 0.05% of HCX (HA GelMa, Alginate and HPMC) and commercial gel containing 0.2% chlorhexidine and unmodified HA (Periokin Hyaluronic 1%, Laboratorios Kin, commercial HA 0.2% CHX) (n=3), like in experiment 5 of section 5.1.

### Results

Individually, in experiments with human gingival fibroblast cells, the cytotoxicity evaluated by lactate dehydrogenase (LDH) activity released into the medium was not observed in the different hydrogels (HA, GelMa, Alginate and HPMC) formulated with and without HCX. On the contrary, the commercial Periokin gel containing 0.2% of CHX showed high cytotoxicity.

The results of FIG. 15 show that all the different hydrogels formulated with 0.05% HCX (HA 0.05% HCX, GelMa 0.05% Formoxanthone A HCX, Alginate 0.05% HCX and HPMC 0.05% HCX) have good antimicrobial properties and are similar or better to the effect produced by a commercial gel containing 0.2% chlorhexidine.

### Example 13: Biocompatibility and Antimicrobial Test of Periodontal Gels Using Human Oral Epithelium, Human Gingival Epithelium and P. Gingivalis

### Material and Methods

To determine the biocompatibility on Human Oral Epithelium and Human Gingival Epithelium of commercial periodontal gels compared to modified HA hydrogel with HCX, an in vitro model histologically similar to the outer cell layers of the oral epithelium and in vitro model histologically similar to the outer cell layers of the human gum were used after exposure to the different gels and using a MTT viability test as an indicator of biocompatibility.

To determine the antimicrobial activity of commercial periodontal gels compared to modified HA hydrogel with HCX, the P. gingivalis strain of bacteria characteristic of periodontal disease was used.

13.1. Treatment with Different Periodontal Gels.

Four different commercial gels (A to D) and a modified HA gel with 0.05% of 9-hydroxycalabaxanthone (HCX) (E) were used in the present study:
A. PerioKIN Hyaluronic 1% (Laboratorios Kin, Barcelona, Spain), which contains 0.2% of chlorhexidine (CHX) and 1% hyaluronic acid (HA), identified as PerioKIN HA 0.2% CHX.
B. Perio-Aid Gel Bioadhesive (Dentaid SL, Barcelona, Spain), which contains 0.2% of CHX and 0.2% of HA, identified as Perio-Aid HA 0.2% CHX.
C. Bexident post gel periodontal (ISDIN, Barcelona, Spain), which contains 0.2% of chlorhexidine (CHX) and 0.5% of chitosan, identified as Bexident Chitosan 0.2% CHX.
D. Lacer Mucorepair gel (Lacer SA, Barcelona, Spain), which contains 0.2% of enoxolone, 0.2% of HA, and 0.3% of asiaticoside, identified as Mucorepair 0.2% Enoxolone.
E. Modified HA hydrogel with 0.05% HCX was synthesized like in experiment 2, section 2.3, identified as HA 0.05% HCX).

### 13.2. Reconstructed Human Oral Epithelium and Human Gingival Epithelium.

The SkinEthic™ Human Oral Epithelium (HOE) tissue model (EpiSkin, Lyon, France) was used as in vitro model to perform the biocompatibility test. The HOE model is composed of TR146 cells (derived from a squamous cell carcinoma of the buccal mucosa) cultivated on an inert polycarbonate filter at the air liquid interface in a chemically defined medium. This model forms an epithelial tissue devoid of stratum corneum, resembling histologically to the mucosa of the oral cavity.

The SkinEthic™ Human Gingival Epithelium (HGE) was used as in vitro model to perform the biocompatibility test. HGE model is composed of normal human gingival cells cultivated on an inert polycarbonate filter at the air liquid interface in a chemically defined medium. This model is histologically like the outer cell layers of the human gum.

The inserts containing the HOE and HE (size 0.5 cm²) were shipped at room temperature in a multi-well plate filled with an agarose-nutrient solution in which they are embedded. Upon arrival, the tissues were processed following the manufacturer's protocol. Briefly, the HOE and HGE tissues were removed from the agarose-nutrient solution and placed in a plate previously filled with the SkinEthic™ Maintenance Medium (EpiSkin, Lyon, France) at room temperature. The tissues were incubated in a cell incubator at 37° C., 5% CO₂, and saturated humidity for 48 hours.

13.3. HOE and HGE Treated with Different Antiseptic Skin Gels.

After 48 hours of incubation, the HOE and HGE tissues were surface treated with the different periodontal gels or with positive or negative control for 3 hours. HOE and HGE tissues were assessed for tissue viability (MTT assay) to culture media, like in experiment 4, section 4.4. For each time-point three tissue samples per group were used. 50 μL of the different periodontal gel was inserted atop of the reconstructed HOE samples and incubated at 37° C. for 3 h. For negative control (C−), 1:1 milliQ water dilution in PBS was used and for positive control (C+), a 10% SDS (Sigma-Aldrich, St. Louis, MO) solution was prepared in water, and diluted 1:1 in PBS with a final concentration of 5% SDS. For both controls, 50 μL of these solutions were inserted atop of the reconstructed HOE and HGE samples and run in parallel to the test samples.

13.4. Antimicrobial Test of Antiseptic Skin Gels on P. gingivalis.

The bacteria P. gingivalis was cultured and treated with different commercial periodontal gels and HA formulated with 0.05% HCX. The gels were tested at a concentration of 5% (v/v), the experiment was run like in experiment 1 of section 1.6.

### Results

The results of FIG. 20A shows that the modified HA gel with 3 mM of HCX presents very good biocompatibility in a HOE tissue, similar to an untreated oral control tissue (C−). However, the commercial gels containing 0.2% chlorhexidine with unmodified hyaluronic acid or chitosan (Periokin HA 0.2% CHX, Perio-Aid HA 0.2% CHX and Bexident Chitosan 0.2% CHX), and the commercial gel containing 0.2% of Enoxolone (Mucorepair 0.2% Enoxolone) presented toxic effects on the HOE, below 50% of viability. Moreover, FIG. 20B shows that Periokin HA 0.2%, Perio-Aid HA 0.2% CHX, Bexident Chitosan 0.2% CHX and the modified HA gel with 0.05% of HCX presents very good biocompatibility in a HGE tissue, similar to an untreated oral control tissue (C−). However, the commercial gel Mucorepair 0.2% Enoxolone presented toxic effects on the GTE, below 64% of viability.

In addition, the results of FIGS. 20C and D show that all periodontal gels have a high antibacterial activity against P. gingivalis, a characteristic bacterium of periodontal diseases.

### Example 14: Biocompatibility and Antimicrobial Test of Antiseptic Skin Gels Using Reconstructed Human Epidermis and S. Aureus

### Material and Methods

To determine the biocompatibility on Reconstructed Human Epidermis of commercial antiseptic skin gels compared to modified HA hydrogel with HCX, an in vitro model histologically similar to the in vivo human epidermis was used after exposure to antiseptic skin gels and using a MTT viability test as an indicator of biocompatibility.

To determine the antimicrobial activity of commercial antimicrobial skin gels compared to modified HA hydrogel with HCX, the S. aureus strain of bacteria characteristic of skin infections was used.

14.1. Treatment with Different Antiseptic Skin Gels

Three different commercial gels (A to C) and modified HA gel with 0.05% of 9-hydroxycalabaxanthone (HCX) (D) were used in the present study:
A. Betadine 10% topical gel (Mylan, West Virginia, United States), which containing povidone iodine as an antiseptic, identified as Betadine.
B. Cristalmina film 1% topical gel (Laboratorios SALVAT, Barcelona, Spain), which contains 1% of CHX as an antiseptic, identified as Cristalmina.
C. Prontosan® Gel (B. Braun, Melsungen, Germany), which contains 0.1% Undecylenamidopropyl Betaine, 0.1% Polyhexanide as antiseptic, identified as Prontosan.
D. Modified HA hydrogel with and without 0.05% HCX was synthesized like in experiment 2, section 2.3, identified as HA 0.05% HCX.

### 14.2. Reconstructed Human Epidermis

The EpiSkin™ Small/Reconstructed Human Epidermis (EpiSkin, Lyon, France) was used as in vitro model to perform the biocompatibility test. The model is composed of normal human keratinocytes cultured on a collagen matrix at the air-liquid interface, and it is histologically similar to the in vivo human epidermis. This model exists at different stages of maturity, in this case age was day 13.

The inserts containing the RHE (size 0.38 cm²) were shipped at room temperature in a multi-well plate filled with an agarose-nutrient solution in which they are embedded. Upon arrival, the tissues were processed following the manufacturer's protocol. Briefly, the Episkin™ tissues were removed from the agarose-nutrient solution and placed in a plate previously filled with the EpiSkin™ Maintenance Medium (EpiSkin, Lyon, France) at room temperature. The tissues were incubated in a cell incubator at 37° C., 5% CO2, and saturated humidity for 48 hours.

14.3. Treated with Different Antiseptic Skin Gels.

After 48 hours of incubation, the Episkin™ tissues were surface treated with antiseptic skin gels or with negative control for 3 hours. RHE tissues were assessed for tissue viability (MTT assay) to culture media, like in experiment 4, section 4.4. For each time-point three tissue samples per group were used. 40 μL of the different skin gel was inserted atop of the reconstructed RHE samples and incubated at 37° C. for 3 h. For negative control (C−), 1:1 milliQ water dilution in PBS was used. For negative control, 40 μL of the solution was inserted atop of the Episkin™ samples and run in parallel to the test samples.

14.4. Antimicrobial Test of Antiseptic Skin Gels on S. aureus.

The bacteria S. aureus was cultured and treated with different commercial antiseptic gels and HA hydrogel with 3 mM HCX. The gels were tested at a concentration of 5% (v/v), and the experiment was run like in experiment 7, section 7.1.

### Results

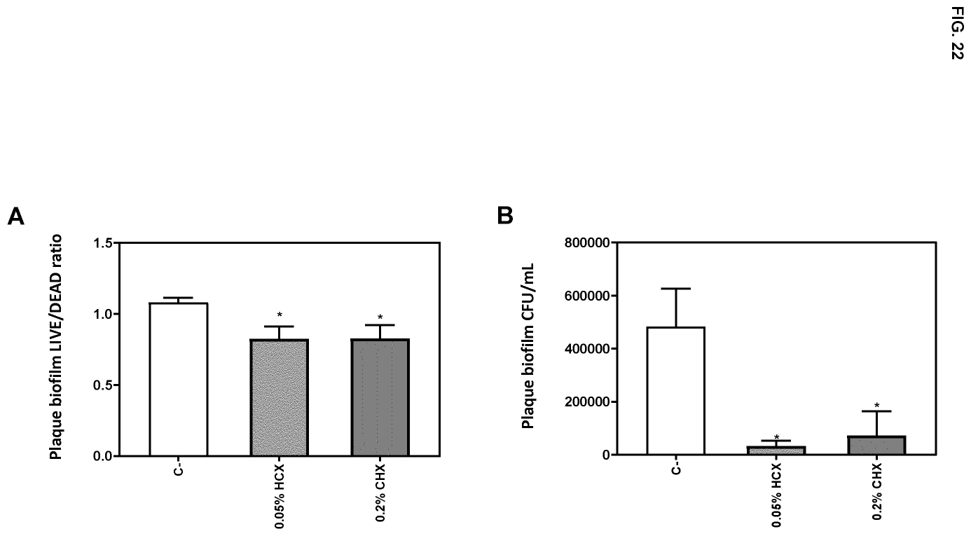

The results of FIG. 21A show that the modified HA gel with 0.05% of HCX, Betadine gel and Prontosan gel presented good biocompatibility on Episkin™ tissue, similar to an untreated epidermis control tissue (C−). However, the commercial Cristalimina gel containing 1% of CHX had a toxic effect on the tissue, with only 27% of viability. Moreover, the results of FIGS. 21B and C show that all the antiseptic skin gels present a high antibacterial activity against S. aureus.

### Example 15: In Vitro Study of Bacterial Plaque Formation

### Material and Methods

This is an in vitro study using pooled human saliva as a test substrate to compare the ability of 9-hydroxycalabaxanthone (HCX) versus chlorhexidine (CHX) and control to prevent or reduce plaque biofilm buildup.

### 15.1. Bacterial Plaque Formation and Treatment

Human saliva from different donors was collected and pooled. 15 ml of the pooled saliva supplemented with 0.1% sucrose was pipetted into sterile 40 ml containers and then a glass microscope slide was submerged below 2 cm of saliva in each container and incubated at 37° C. for bacterial biofilm formation. Twice a day the slide was immersed in TSB culture medium. On days 2 and 3, the biofilm was treated with different groups: (1) a solution containing 0.05% 9-hydroxycalabaxanthone (HCX) (n=6), (2) a solution containing 0.2% chlorhexidine (CHX) (n=6), and a solution containing water (C−) (n=6), by immersing the slides for 2 min in the test of the assigned product. On day 4, the biofilms were dipped three times in saline, placed in 5 mL of sterile saline, and the surfaces of the slides were gently scraped with a sterile scraper to collect adherent bacteria. The removed biofilms were subjected to sonication using three 15 s pulses with a power of 7 W. The homogenized bacterial suspension was used to determine the bacterial viability both by quantification of colony-forming units (CFU) per mL and by LIVE/DEAD assay.

For the Bacterial viability assay, an aliquot (0.1 mL) of the homogenized bacterial suspension was serially diluted and plated on TSB agar. The plates were incubated in 5% CO₂ at 37° C. for 48 h, and then the number of CFU/mL was determined. Moreover, an aliquot (0.1 mL) of the homogenized bacterial suspension was used to determine the Live/Dead ratio of bacteria using the LIVE/DEAD BacLight bacterial viability kit (Invitrogen, Thermo Fisher Scientific, Waltham, MA, USA), following manufacturer's instructions.

### Results

FIG. 22 shows that the treatment with 0.05% HCX reduced plaque biofilm bacteria formation with results equal to 0.2% chlorhexidine, the positive control.

### Example 16: Evaluation of the Antimicrobial Activity of HCX on Cutibacterium acnes

### Material and Methods

11.1. Culture and Proliferation Assay of C. acnes.

C. acnes is a lipophilic anaerobic Gram-positive bacterium. It is part of the commensal flora of the skin, colonizing pilous follicles and sebaceous glands, and may also be found in the mucosa of the mouth, nose, urogenital tract, and large intestine. Pathogenicity has been demonstrated in many infections: cutaneous, digestive, and cardiovascular, and in orthopedic surgery and particularly in relation to implants. This example is provided to give an example for its use topically in the skin to treat acne.

C. acnes 5684 (CECT, Valencia, Spain) was grown on Tryptic soy broth (TSB) (Scharlab), supplemented with 0.5 g/I L-Cysteine hydrochloride (Thermo Fisher Scientific), 1.0 mg/I hemin (Thermo Fisher Scientific) and 5.0 mg/I glucose (Scharlab) under anaerobic conditions (10% H2, 10% CO2 and 80% N2) achieved with an Oxoid Anaerogen™ sachet (Thermo Fisher Scientific) at 37° C. for 48 h. After overnight incubation, 1 mL of C. acnes was incubated with different concentrations of CHX (0.01, 0.005, 0.003, 0.001%) and HCX (0.003, 0.002, 0.001, 0.0002%) for 24 hours at 37° C. under anaerobic conditions achieved with an Oxoid Anaerogen™ sachet. Bacterial suspension without treatment served as negative control (C−). The optical density (OD) was measured at 600 nm at 0 h and 24 h to determine bacterial proliferation (PowerWave Ht). The bacterial growth rate (μ) was calculated during the exponential growth phase following the equation ln ODt−ln OD0=μ×(t−t0) (n=4). The live/Dead ratio of bacteria was determined using the LIVE/DEAD BacLight bacterial viability kit (Invitrogen), following the manufacturer's instructions. The assays were carried out in triplicate.

### Results

Cutibacterium acnes predominates in sebaceous sites, is critical in the regulation of skin homeostasis and prevents colonisation from other harmful pathogens, it can also act as an opportunistic pathogen in acne vulgaris.

In this example (FIG. 23), we show that a concentration of 0.0002% of HCX incubated for 24 h on C. acnes is sufficient to eliminate 96% of bacteria, which is similar to the effectivity of the most common antiseptic used for the skin.

### Example 17: Evaluation of Different Concentrations of Xanthones on Fibroblast Viability and on Antibacterial Activity

For the use of 9-hydroxycalabaxanthone and other xanthones, a proper balance between antibacterial activity for a specific pathogen/disease and cytotoxicity of skin/oral tissue is needed for the desired technical advantage but difficult to predict.

### Material and Methods

16.1. Evaluation of the antimicrobial activity of concentration of different xanthones on S. aureus and of the cytotoxicity thereof in fibroblasts Different concentrations of xanthones: 9-hydroxycalabaxanthone (HCX), alpha-mangostin (α-MG), norathryol (NRT), 1,3,7-trihydroxy-2-prenylxanthone (PX), Formoxanthone A (FXA) (0.4 μM, 4 μM, 19 μM, 39 μM and 77 μM, which corresponded to different % according to their molecular weight) were used for studies with fibroblasts and S. aureus. Treated cells without the biomolecule served as negative control (C−).

Immortalized human gingival fibroblasts were cultured like in experiment 1 of section 1.2. The cells were treated with different concentration of xanthones in a fibroblast medium for 24 hours (n=4). Cell cytotoxicity was measured, like in experiment 1 of section 1.4.

The bacteria S. aureus was cultured and treated with different concentration of xanthones, like in experiment 7 of section 7.1.

### Results

α-Mangostin (aMG) and 9-hydroxycalabaxanthone (HCX) are two xanthones from the biprenyl xanthone group. They have a similar structure with two prenyl substituents and one methoxy group. aMG has 3 hydroxyl groups in its structure while HCX has only 2. The great structural difference between these two molecules lies in the cyclization of one of the prenyl groups in the case of HCX.

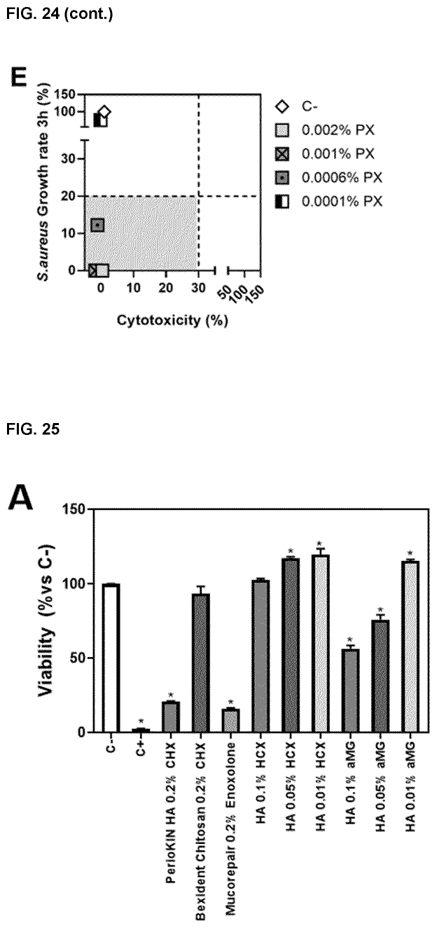

In this example (FIGS. 24A and 24B), we show that a concentration of 0.002% and 0.001% of HCX and a concentration of 0.0002% and 0.001% of aMG incubated for 3 h on S. aureus is sufficient to eliminate more than 80% of bacteria and these concentrations are no cytotoxic on fibroblasts (cytotoxicity is lower than 30%). The major structural difference between these two molecules lies in the cyclisation of one of the prenyl groups in the case of HCX, which should increase cytotoxicity and decreased antibacterial activity. However, this was not the case, as surprisingly 9-hydroxycalabaxanthone can be applied to cells and tissues up to 0.002% when formulated in solution while aMG was already toxic at this concentration.

NRT and HCX have the characteristic skeleton of xanthones but have variations in the number of substituents on their aromatic rings. In the case of NRT has only four alcohol groups (−OH) in positions 1, 3, 6 and 7. As for HCX has two prenyl groups, one of them cyclized in positions 2 and 3, two alcohol groups and a methoxy group. As shown in FIG. 24C shows all the concentrations of NRT tested were biocompatible for the cells, but none of them were antibacterial. Therefore, prenylation seems to be an important substituent for the antibacterial effect of xanthones.

FXA and HCX have the characteristic skeleton of xanthones but the substituents on their aromatic rings vary. They have a similar structure with two prenyl substituents, but HCX has one methoxy group and 2 hydroxyl groups, while FXA has 3 hydroxyl groups in its structure and without a methoxy group. FIG. 24 D, shows that only 0.001% FXA has antibacterial effect without cytotoxicity, thus, the position of prenyl substituents and has also an important impact on the effects of the xanthones.

In the case of PX (FIG. 24 E), it has only three alcohol groups (−OH) and one prenyl group and showed good antibacterial and biocompatibility from 0.0006 to 0.002%.

In conclusion, we provide in the present example, specific concentration of other xanthone compounds with a bactericidal effect but preserved biocompatibility of cells/tissues, demonstrating that prenylation of the xanthone skeleton is the most important substituent to achieve both effects. However, not only the presence of the prenyl group is important but also its position. Therefore, a proper balance is necessary but difficult to envisage. The bactericidal effect was comparable to that of known bacteriostatic agents, for example, chlorhexidine, with the disadvantage that chlorhexidine is toxic to cells/tissues.

### Example 18: Biocompatibility and Antimicrobial Test of Periodontal Gels Formulated with HCX and aMGusing Human Gingival Epithelium and S. Mutans

### Material and Methods

To determine the biocompatibility on Human Gingival Epithelium (HGE) of commercial periodontal gels compared to modified HA hydrogel with different concentrations of 9-hydroxycalabaxanthone (HCX) and α-Mangostin (α-MG), an in vitro model histologically similar to the outer cell layers of the human gum were used after exposure to the different gels and using a MTT viability test as an indicator of biocompatibility.

To determine the antimicrobial activity of commercial periodontal gels compared to modified HA hydrogel with different concentrations of HCX and α-MG, the S. mutans strain of bacteria characteristic of oral disease was used.

18.1. Treatment with Different Periodontal Gels.

Three different commercial gels (A to C) and a modified HA gel with 0.1%, 0.05% and 0.01% of 9-hydroxycalabaxanthone (HCX) (D) and modified HA gel with 0.1%, 0.05% and 0.01% of α-Mangostin (α-MG) (E) were used in the present study:
A. PerioKIN Hyaluronic 1% (Laboratorios Kin, Barcelona, Spain), which contains 0.2% of chlorhexidine (CHX) and 1% hyaluronic acid (HA), identified as PerioKIN HA 0.2% CHX.
B. Bexident post gel periodontal (ISDIN, Barcelona, Spain), which contains 0.2% of chlorhexidine (CHX) and 0.5% of chitosan, identified as Bexident Chitosan 0.2% CHX.
C. Lacer Mucorepair gel (Lacer SA, Barcelona, Spain), which contains 0.2% of enoxolone, 0.2% of HA and 0.3% of asiaticoside, identified as Mucorepair 0.2% Enoxolone.
D. Modified HA hydrogel with 0.1%. 0.05% and 0.01% HCX were synthesized like in experiment 2, section 2.3, identified as HA 0.1% HCX, HA 0.05% HCX, and HA 0.001% HCX).
E. Modified HA hydrogel with 0.1%. 0.05% and 0.01% aMG were synthesized like in experiment 2, section 2.3, identified as HA 0.1% α-MG, HA 0.05% aMG and HA 0.001% α-MG).

### 18.2. Reconstructed Human Gingival Epithelium.

The inserts containing HGE (size 0.5 cm²) were shipped at room temperature in a multi-well plate filled with an agarose-nutrient solution in which they are embedded. Upon arrival, the tissues were processed following the manufacturer's protocol. Briefly, the HGE tissues were removed from the agarose-nutrient solution and placed in a plate previously filled with the SkinEthic™ Maintenance Medium (EpiSkin, Lyon, France) at room temperature. The tissues were incubated in a cell incubator at 37° C., 5% CO2, and saturated humidity for 48 hours.

18.3. HGE Treated with Different Antiseptic Skin Gels.

After 48 hours of incubation, the HGE tissues were surface treated with the different periodontal gels or with positive or negative control for 24 hours. HGE tissues were assessed for tissue viability (MTT assay), like in experiment 4, section 4.4. For each time-point three tissue samples per group were used. 50 μL of each periodontal gel were inserted atop of the reconstructed HGE samples and incubated at 37° C. for 24 h. For negative control (C−), 1:1 milliQ water dilution in PBS was used and for positive control (C+), a 10% SDS (Sigma-Aldrich, St. Louis, MO) solution was prepared in water, and diluted 1:1 in PBS with a final concentration of 5% SDS. For both controls, 50 μL of these solutions were inserted atop of the reconstructed HGE samples and run in parallel to the test samples.

18.4. Antimicrobial Test of Antiseptic Skin Gels on S. mutans

The bacteria S. mutans was cultured and treated with different commercial periodontal gels and HA formulated with 0.1%, 0.05%, and 0.01% HCX and aMG for 24 h. The gels were tested at a concentration of 5% (v/v), and the experiment was run like in experiment 8 of section 8.1.

### Results

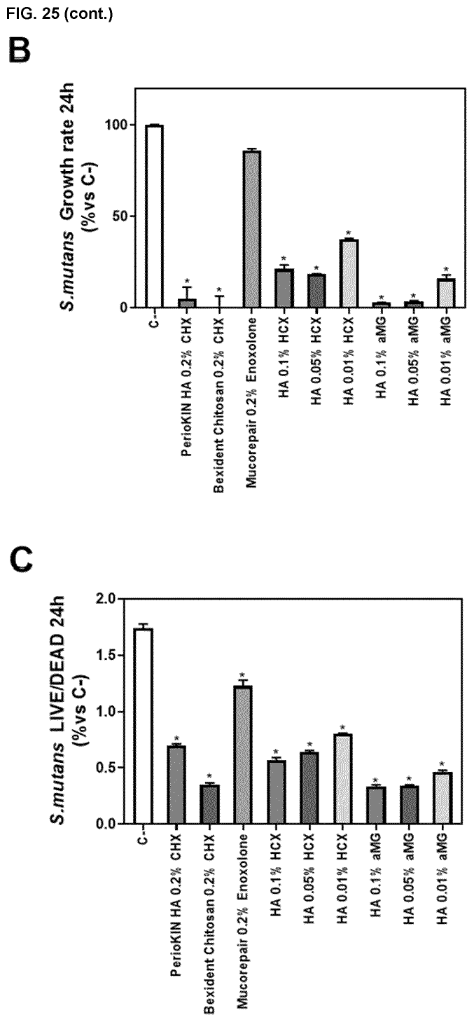

The results of FIG. 25A show that the modified HA gel with 0.1%, 0.05%, and 0.01% of HCX, the modified HA gel with 0.01% of α-MG, and the commercial Bexident Chitosan 0.2% CHX gel presented good biocompatibility on HGE tissue after 24 hours, similar or improved compared to an untreated control tissue (C−). However, the modified HA gel with 0.1% and 0.05% of α-MG, the commercial Periokin HA 0.2% CHX gel, and Mucorepair 0.2% Enoxolone gel had a toxic effect on the tissue, below 76% of viability.

Moreover, the results of FIGS. 25 B and C show that the modified HA gel with 0.1%, 0.05% and 0.01% of HCX, the modified HA gel with 0.1%, 0.05%, and 0.01% of α-MG, the commercial Periokin HA 0.2% gel and the commercial Bexident Chitosan 0.2% CHX gel presented a high antibacterial activity against S. mutans. However, the commercial Mucorepair 0.2% Enoxolone gel reduced bacterial growth by only 14%.

Thus, all HA gels containing different concentrations of HCX had high biocompatibility and antibacterial effect, and also 0.01% of aMG formulated in a HA gel. This showed better outcomes than commercial periodontal gels formulated with CHX or enoxolone.

1. A method of preventing or treating a disease of microbial origin comprising administering via tropical administration to a subject in need thereof a composition comprising a compound having formula I: 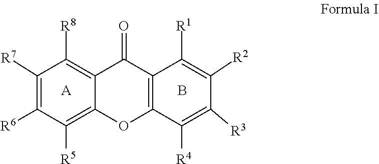
wherein
R1 is a hydroxyl group or a hydrogen atom;
R2 is a prenyl group or a hydrogen atom, wherein when R2 is a prenyl group and R3 or R1 are a hydroxyl group or a methoxy group, R2 and R3 or R2 and R1 optionally link together forming a ring;
R3 is a hydroxyl group, a methoxy group or a hydrogen atom;
R4 is a prenyl group or a hydrogen atom;
R5 is a hydroxyl group, a prenyl group, a methoxy group or a hydrogen atom;
R6 is a hydroxyl group, a methoxy group or a hydrogen atom;
R7 is a hydroxyl group, a prenyl group, a methoxy group or a hydrogen atom; and
R8 is a prenyl group or a hydrogen atom, wherein when R8 is a prenyl group and R7 is a hydroxyl group or a methoxy group, R8 and R7 optionally link together forming a ring;
or any salts thereof.

2. The method of claim 1, wherein
R1, R3 and R6 are independently selected from a hydroxyl group or a hydrogen atom;
R2 and R8 are independently selected from a prenyl group or a hydrogen atom; wherein when R2 is a prenyl group and R3 or R1 are a hydroxyl group or a methoxy group, R2 and R3 or R2 and R1 optionally link together forming a ring; and wherein when R8 is a prenyl group and R7 is a hydroxyl group or a methoxy group, R8 and R7 optionally link together forming a ring;
R7 and R5 are independently selected from a methoxy group or a hydrogen atom; and
R4 is a hydrogen atom:
or any salts thereof.

3. The method of claim 1, wherein the compound has formula II: 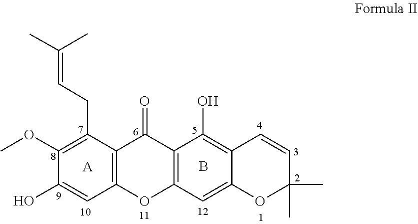
or any salts thereof.

4. The method of claim 1, wherein the composition is in the form of a hydrogel, wherein the compound is entrapped in the hydrogel, and the compound is present in the composition in an amount of from 0.0002% to 0.2% by weight/volume of the total composition.

5. The method of claim 4, wherein the compound is present in the composition in an amount of from 0.005% to 0.05% by weight/volume of the total composition.

6. The method of claim 1, wherein the composition is in the form of a solution and the compound is present in the composition in an amount of from 0.00001% to about 0.1%.

7. The method of claim 6, wherein the compound is present in the composition in an amount of from 0.0002% to 0.002% by weight/volume of the total composition.

8. The method of claim 1, wherein the composition is in the form of a solution or in aqueous form, wherein the compound is present in said compositions in an amount of from 0.001% to 0.002% by weight/volume of the total composition.

9. The method of claim 1, wherein the disease of microbial origin in a skin is selected from Staphylococcus aureus, Staphylococcus epidermidis, Cutibacterium acnes, Streptococcus pyogenes or Epidermolysis bullosa.

10. The method of claim 1, wherein the disease is selected from the list consisting of acne, impetigo, psoriasis, erysipelas, necrotizing fasciitis, cellulitis, folliculitis, furuncles and carbuncles, and scalded skin syndrome.

11. The method of claim 4, wherein the diseases of microbial origin is selected from the list consisting of periodontal diseases, plaque, caries, halitosis, gingivitis, mucositis, mycosis, and oral aphthous ulcers.

12. (canceled)

13. The method of claim 3, wherein the composition is in the form of a hydrogel comprising water, a hyaluronic acid derivative and 9-hydroxycalabaxanthone or a salt thereof (HCX), wherein:
a. the hyaluronic acid derivative comprises hyaluronic acid, or a salt thereof, of molecular weight comprised between 50,000 and 3,500,000 Da;
b. the concentration of said hyaluronic acid derivative or salt thereof is comprised between 0.001% and 5%; and
c. the 9-hydroxycalabaxanthone or a salt thereof (HCX) is entrapped in the hydrogel and has a concentration comprised between 1 mM and 3 mM.

14. The method of claim 13, wherein the hyaluronic acid is at temperatures inducing gelling (physical crosslinking) and/or covalently crosslinked by chemically functionalized groups reactive to polymerization.

15. The method of claim 13, wherein the hyaluronic acid or derivative thereof is chemically functionalized to become reactive to polymerization or crosslinking by nucleophilic addition reaction.

16. The method of claim 15, wherein the hyaluronic acid or derivative thereof is crosslinked by the mix of two lots of hyaluronic acid where the first of them is functionalized with adipic acid dihydrazide and the second of them is functionalized with aldehyde groups by the oxidation of vicinal hydroxyl groups.
